(19) United States
(12) Patent Application Publication
(10) Pub. No.: US 2023/0321124 A1
(43) Pub. Date: Oct. 12, 2023

(54) ROR gamma t Inhibitors and Topical Uses Thereof

(71) Applicant: DERMIRA, INC., Menlo Park, CA (US)

(72) Inventors: Jamie L. Harden, Menlo Park, CA (US); Delphine Imbert, Menlo Park, CA (US)

(21) Appl. No.: 18/007,940

(22) PCT Filed: Jun. 29, 2021
(86) PCT No.: PCT/US2021/039560

§ 371 Date: Dec. 2, 2022

Related U.S. Application Data
(60) Provisional application No. 63/046,560, filed on Jun. 30, 2020.

(51) Int. Cl. A61K 31/695; A61K 47/10; A61K 47/22; A61K 47/14; A61K 47/38; A61K 47/26; A61K 47/12; A61K 9/06; A61K 47/32; A61K 9/00

(52) U.S. Cl. CPC A61K 31/695; A61K 47/10; A61K 47/22; A61K 47/14; A61K 47/38; A61K 47/26; A61K 47/12; A61K 9/06; A61K 47/32; A61K 9/0014

(57) ABSTRACT

The present disclosure is directed to use of RORγt inhibitors in the treatment of autoimmune disorders, e.g., autoimmune disorders of the skin. This invention is also directed to pharmaceutical compositions comprising an RORγt inhibitor and a pharmaceutically acceptable carrier for topical administration.

### CROSS-REFERENCE TO RELATED APPLICATIONS

This application claims the benefit of U.S. Provisional Application No. 63/046,560, filed Jun. 30, 2020, the disclosure of which is incorporated herein be reference.

### BACKGROUND

The retinoic acid receptor-related orphan nuclear receptor (ROR) RORγ and its isoform RORγt play a major role in regulation of a variety of biological systems. RORγt is known to play a central role in immune system development, homeostasis, and responses to microbial pathogens. RORγt is required for the differentiation of Th17 cells, a subset of T helper cells that protect the host from infection by secreting inflammatory cytokines such as IL-17, IL-17A, IL-17F, IL-22, and TNFα. These cytokines are signaling proteins that have been shown to be essential in regulating numerous immune responses, including inflammatory responses to antigens. Th17 cells have also recently been shown to have important roles in activating and directing immune responses in a variety of autoimmune diseases, such as experimental autoimmune encephalomyelitis (EAE), collagen-induced arthritis (CIA), inflammatory bowel disease (IBD), and cancer. Th17 cells have also been implicated in asthma, psoriasis, rheumatoid arthritis, multiple sclerosis, atopic dermatitis and Crohn's disease. Additionally, it has been shown that mice defective for expression of RORγt lack Th17 cells and are resistant to a variety of autoimmune diseases, and that the absence of Th17-inducing microbiota in the small intestine of mice alters the Th17: regulatory T (Treg) cell balance with implications for intestinal immunity, tolerance, and susceptibility to inflammatory bowel diseases.

For example, it has been well established that psoriasis vulgaris is mediated primarily by Th17 polarized T-cells. Biologics targeting the Th17 pathway have proven extremely efficacious in the treatment of this disease. However, biologics are expensive and systemic treatments are typically reserved for patients with severe forms of the disease. RORγt is the master transcription factor for Th17 cell polarization and subsequent Th17 associated cytokine production. RORγt knockout mice are protected against many autoimmune diseases caused by Th17 cells, including psoriasis-like models. Furthermore, pharmacologic blocking RORγt in both murine and human cells and tissues results in inhibition of Th17 polarization and Th17 associated cytokines. Importantly, oral RORγt inhibitors have been tested in humans and found to significantly inhibit IL-17A protein production, demonstrating the role of this key Th17 transcription factor in humans in vivo.

Moderate-severe psoriasis patients are typically administered highly effective biologics, but the mild-moderate psoriasis patient population does not have access to these Th17-specific biologics. First line treatment for mild-moderate patients include topical corticosteroids (TCS), calcipotriol, anthralin, or photochemotherapy, but treat to varying degrees of success and adverse event profiles. Adverse events related to chronic use of steroids make this treatment option less appealing to physicians and mild-moderate patients that do not qualify for biologics, and thus patients often prefer non-steroidal creams. Options such as Vitamin D, while safer, are not as efficacious as topical corticosteroids. Thus, a non-steroidal topical treatment that demonstrates superior or comparable efficacy to TCS that does not carry the same adverse event profile as the known therapeutics on the market is desirable.

### SUMMARY

Described herein are topical compositions comprising RORγt inhibitors and methods for using the RORγt inhibitors for the treatment of autoimmune disorders, such as psoriasis.

Therefore, in a first aspect, the present disclosure provides for a topical composition comprising a pharmaceutically effective amount of a RORγt inhibitor (e.g., a RORγt inhibitor of the present disclosure); a dermatologically acceptable carrier; a humectant; and a preservative.

In a second aspect, the present disclosure provides for a method for treating an autoimmune disorder, the method comprising topically administering to a subject in need thereof a topical composition having a therapeutically effective amount of a RORγt inhibitor (e.g. a RORγt inhibitor according to the present disclosure); and a dermatologically acceptable excipient.

### BRIEF DESCRIPTION OF THE FIGURES

FIG. 1 illustrates the performance of three RORγt inhibitors according to the present disclosure in various formulations in sRICA. The prepared formulations are summarized in Table 1 and include creams (Formulations 3-7), aqueous gels (Formulation 8), non-aqueous gels (Formulations 9-10) and PEG based ointments (Formulation 11).

FIG. 2 illustrates the performance of RORγt inhibitors according to the present disclosure in various vehicle formulations in sRICA. The vehicles used are summarized in Tables 5-7.

FIG. 3 illustrates the performance of RORγt inhibitors according to the present disclosure in various formulations in sRICA at two time points.

### DETAILED DESCRIPTION

Provided herein are topical compositions for treating autoimmune disorders, e.g., autoimmune disorders characterized by inflammation. In particular, the pharmaceutical compositions include compounds that are inhibitors of receptor-related orphan nuclear receptor (RORγt). RORγt is the master transcription factor for Th17 cell polarization and subsequent Th17 associated cytokine production. In humans, mutations in Th17 associated genes are highly correlated with autoimmune diseases, including psoriasis. While not wishing to be bound by theory, inhibition of RORγt may attenuate inflammation mediated by Th17, e.g., psoriatic-like skin inflammation.

### RORγt Inhibitors for Use in the Compositions and Methods of the Present Disclosure

In one embodiment, the compounds for use in the presently disclosed compositions and/or methods is a compound of Formula I:

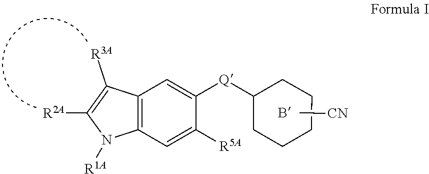

wherein:
R^{1A} is an optionally substituted hydrocarbon group or an optionally substituted hydrocarbon-oxy group;
R^{2A} and R^{3A} are each independently a hydrogen atom, an optionally substituted hydrocarbon group, an optionally substituted hydrocarbon-oxy group, an acyl group, a halogen atom, a cyano group, an optionally substituted hydrocarbon-amino group, an optionally substituted hydrocarbon-sulfanyl group, an optionally substituted hydrocarbon-sulfenyl group, an optionally substituted hydrocarbon-sulfonyl group or a nitro group, or R^{2A} and R^{3A} optionally form, together with the carbon atoms which they are bonded to, an optionally substituted hydrocarbon ring;
R^{5A} is a hydrogen atom or a halogen atom;
Q′ is a bivalent group selected from:

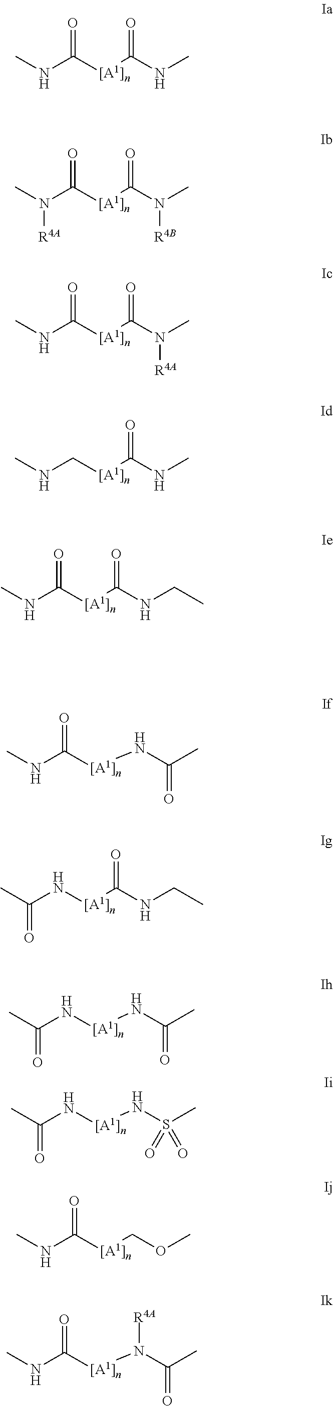

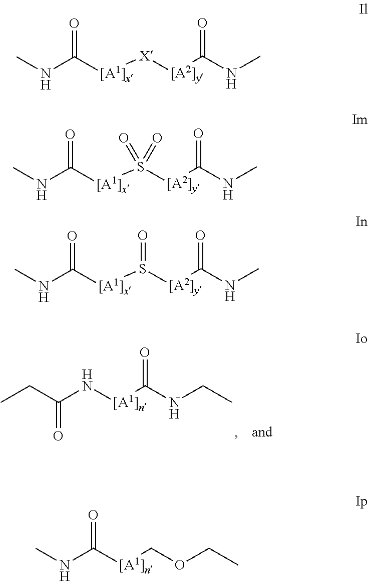

wherein
[A¹] are the same or different and each is a methylene group optionally substituted by substituent(s) selected from a hydroxy group, a phenyl group and an optionally substituted C₁₋₆ alkyl group, wherein the two substituents bonded to the single carbon atom are optionally combined to each other to form a hydrocarbon ring, and [A²] are the same or different and each is a methylene group optionally substituted by substituent(s) selected from a hydroxy group and an optionally substituted C₁₋₆ alkyl group, wherein the two substituents bonded to the single carbon atom are optionally combined to each other to form a hydrocarbon ring, or the methylene group in [A¹] or [A²] is optionally combined to the substituent on the adjacent methylene group to form an optionally substituted hydrocarbon ring,
R^{4A} and R^{4B} are the same or different and each is an optionally substituted hydrocarbon group,
X′ is an oxygen atom, a sulfur atom, or an imino group having an optionally substituted hydrocarbon group or a hydrogen atom,
n is an integer of 1 to 5,
n′ is an integer of 1 to 4,
n″ is an integer of 1 to 3, and
x′ and y′ are each 0 or natural number, and the sum is 0 to 4, and
Ring B′ is a benzene ring optionally having additional substituent(s), or a pyridine ring optionally having additional substituent(s), provided that when R^{5A} is a halogen atom, then Ring B′ is a benzene ring optionally having additional substituent(s), provided that 2-(2-((4-cyanophenyl)amino)-2-oxoethoxy)-N-(9-ethyl-9H-carbazol-3-yl)acetamide and N-(4-cyanophenyl)-N′-(9-ethyl-9H-carbazol-3-yl)-3-methylpentanediamide are excluded)

or a stereoisomer, solvates, tautomers, or pharmaceutically acceptable salts thereof.

In another embodiment, the compounds for use in the presently disclosed compositions and/or methods is a compound of Formula II:

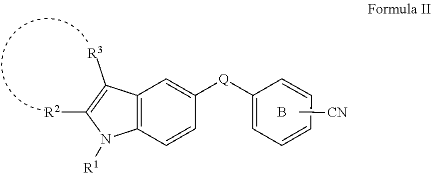

wherein:
R¹ is an optionally substituted hydrocarbon group or an optionally substituted hydrocarbon-oxy group,
R² and R³ are each independently an optionally substituted hydrocarbon group, an optionally substituted hydrocarbon-oxy group, an acyl group, a halogen atom, a cyano group, an optionally substituted hydrocarbon-amino group, an optionally substituted hydrocarbon-sulfanyl group, an optionally substituted hydrocarbon-sulfenyl group, an optionally substituted hydrocarbon-sulfonyl group or a nitro group, or R² and R³ optionally form, together with the carbon atoms which they are bonded to, an optionally substituted hydrocarbon ring,
Q is a bivalent group selected from:

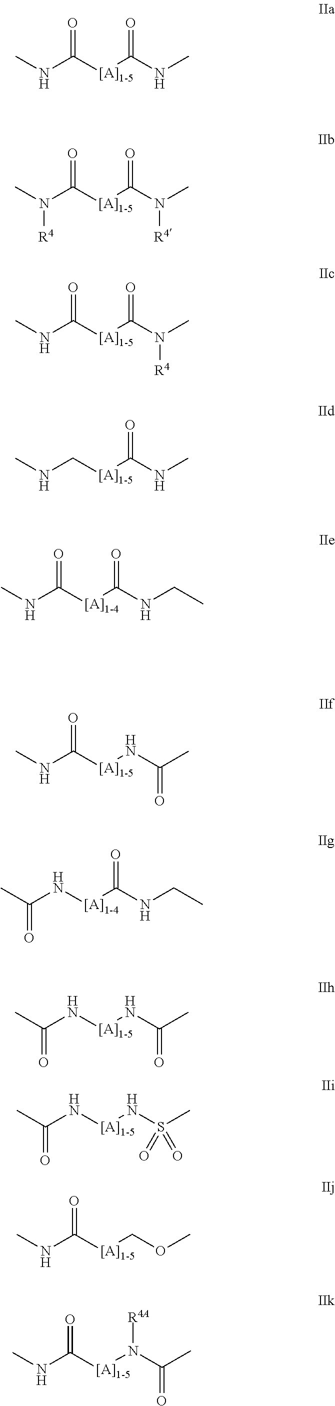

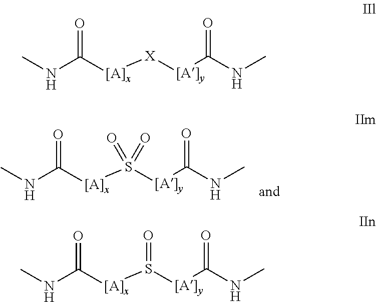

wherein:
[A] are the same or different and each is a methylene group optionally substituted by substituent(s) selected from a hydroxy group and an optionally substituted C₁₋₆ alkyl group, wherein the two substituents bonded to the single carbon atom are optionally combined to each other to form a hydrocarbon ring, and [A′] are the same or different and each is a methylene group optionally substituted by substituent(s) selected from a hydroxy group and an optionally substituted C₁₋₆ alkyl group, wherein the two substituents bonded to the single carbon atom are optionally combined to each other to form a hydrocarbon ring, or the methylene group in [A] or [A′] is optionally combined to the substituent on the adjacent methylene group to form an optionally substituted hydrocarbon ring,
R⁴ and R^{4′} are the same or different and each is an optionally substituted hydrocarbon group,
X is an oxygen atom, a sulfur atom, or an imino group having an optionally substituted hydrocarbon group or a hydrogen atom, and
x and y are each 0 or natural number, and the sum is 0 to 4, and
Ring B is a benzene ring optionally further substituted by substituent(s) excluding cyano,
provided that 2-(2-((4-cyanophenyl)amino)-2-oxoethoxy)-N-(9-ethyl-9H-carbazol-3-yl)acetamide and N-(4-cyanophenyl)-N′-(9-ethyl-9H-carbazol-3-yl)-3-methylpentanediamide are excluded (hereinafter sometimes to be referred to as compound (I)),
or a stereoisomer, solvates, tautomers, or pharmaceutically acceptable salts thereof.

Further RORγt inhibitors for use in the methods of and compositions described herein are described in U.S. Pat. No. 9,120,776 B2 and WO 2013/042782 A1, the disclosures of each of which are incorporated herein by reference in their entireties.

In one embodiment, the compounds for use in the presently disclosed compositions and/or methods is a compound of Formula III:

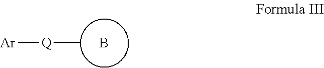

wherein
Ar is the partial structure (1):

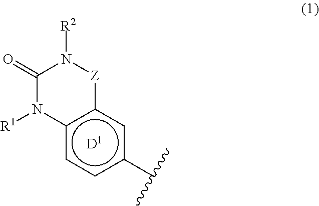

wherein in the partial structure (1),
Z is a carbonyl group or a methylene group,
R¹ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group, an optionally substituted C₇₋₁₆ aralkyl group, an acyl group or a cyano group (excluding a C₁₋₁₂ alkyl group, a C₂₋₁₂ alkenyl group or a C₂₋₁₂ alkynyl group, each substituted by optionally substituted

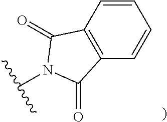 [SMILES: *N1C(=O)c2ccccc2C1=O]

R² is an optionally substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group, an optionally substituted C₇₋₁₆ aralkyl group, an acyl group or a cyano group, and
D¹ is an optionally further substituted 6-membered aromatic ring, the partial structure (2):

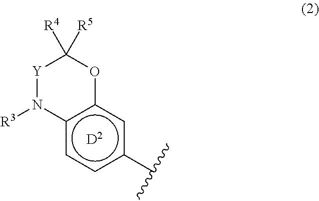

wherein in the partial structure (2),
R³ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group, an optionally substituted C₇₋₁₆ aralkyl group, an acyl group or a cyano group,
Y is an optionally substituted methylene group,
R⁴ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group, an optionally substituted C₇₋₁₆ aralkyl group or an acyl group,
R⁵ is a hydrogen atom or a substituent, or
R⁴ and R⁵ are both methyl groups, or
R⁴ and R⁵ in combination optionally form, together with the carbon atom which they are bonded to, an optionally substituted ring, and
D² is an optionally further substituted 6-membered aromatic ring, or the partial structure (3):

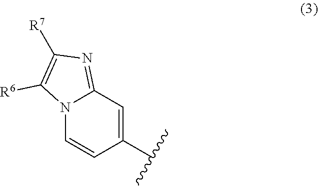

wherein in the partial structure (3),
R⁶ is a C₂₋₁₂ alkyl group, a substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group or an optionally substituted C₇₋₁₆ aralkyl group, and
R⁷ is an optionally substituted C₁₋₁₂ alkyl group, an optionally substituted C₂₋₁₂ alkenyl group, an optionally substituted C₂₋₁₂ alkynyl group, an optionally substituted C₃₋₁₂ cycloalkyl group, an optionally substituted C₃₋₁₂ cycloalkenyl group, an optionally substituted C₆₋₁₄ aryl group or an optionally substituted C₇₋₁₆ aralkyl group,
Q is a bivalent group selected from the group consisting of the following (Ia)-(Ie):

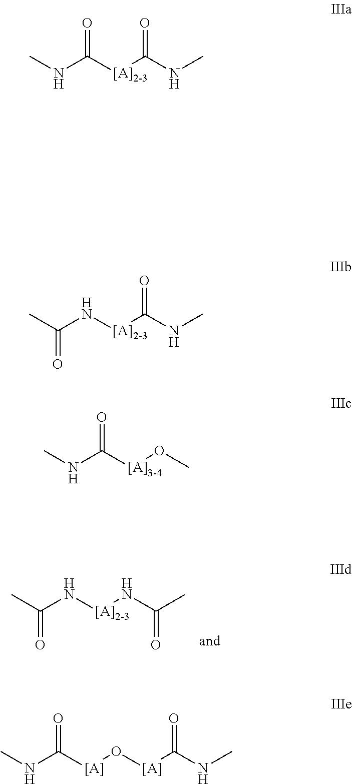

wherein
[A] are the same or different and each is a methylene group optionally substituted by substituent(s) selected from a hydroxy group, an optionally substituted C₁₋₆ alkyl group and a C₆₋₁₄ aryl group, and
B is an optionally substituted ring, provided that
1,2,3,4-tetrahydro-N-[2-[(4-methoxybenzoyl)amino]ethyl]-2,4-dioxo-1-propyl-pyrido[2,3-d]pyrimidine-6-carboxamide,
N-[3-[(5-bromo-2-methylphenyl)amino]-3-oxopropyl]-1-cyclopropyl-1,2,3,4-tetrahydro-2,4-dioxo-pyrido[2,3-d]pyrimidine-6-carboxamide,
N-[3-[(5-bromo-2-methylphenyl)amino]-3-oxopropyl]-1,2,3,4-tetrahydro-2,4-dioxo-1-propyl-pyrido[2,3-d]pyrimidine-6-carboxamide,
1-cyclopropyl-1,2,3,4-tetrahydro-N-[3-[(6-methyl-2-pyridyl)amino]-3-oxopropyl]-2,4-dioxo-pyrido[2,3-d]pyrimidine-6-carboxamide,
1-cyclopropyl-1,2,3,4-tetrahydro-2,4-dioxo-N-[3-oxo-3-(3-pyridylamino)propyl]-pyrido[2,3-d]pyrimidine-6-carboxamide,
1-cyclopropyl-1,2,3,4-tetrahydro-N-[2-[(2H-indazol-3-ylcarbonyl)amino]ethyl]-2,4-dioxo-pyrido[2,3-d]pyrimidine-6-carboxamide,
N-[2-[(2,4-difluorobenzoyl)amino]ethyl]-1,2,3,4-tetrahydro-2,4-dioxo-1-propyl-pyrido[2,3-d]pyrimidine-6-carboxamide,
1,2,3,4-tetrahydro-2,4-dioxo-N-[3-oxo-3-(4-pyridylamino)propyl]-1-propyl-pyrido[2,3-d]pyrimidine-6-carboxamide,
N-[2-[(2-chlorobenzoyl)amino]ethyl]-1,2,3,4-tetrahydro-2,4-dioxo-1-propyl-pyrido[2,3-d]pyrimidine-6-carboxamide,
N-(2-[(4-chlorobenzoyl)amino]ethyl)-1-cyclopropyl-2,4-dioxo-1,2,3,4-tetrahydropyrido[2,3-d]pyrimidine-6-carboxamide,
N-[2-(benzoylamino)ethyl]-1-cyclopropyl-2,4-dioxo-1,2,3,4-tetrahydropyrido[2,3-d]pyrimidine-6-carboxamide, and
1-cyclopropyl-N-[2-[(3-fluoro-4-methylbenzoyl)amino]ethyl]-1,2,3,4-tetrahydro-2,4-dioxo-pyrido[2,3-d]pyrimidine-6-carboxamide
   are excluded,

or a stereoisomer, solvates, tautomers, or pharmaceutically acceptable salts thereof.

Further RORγt inhibitors for use in the methods of and compositions described herein are described in U.S. Pat. No. 9,834,520 B2 and WO 2014/142255A1, the disclosures of each of which are incorporated herein by reference in their entireties.

In one embodiment, the compounds for use in the presently disclosed compositions and/or methods is a compound of Formula IV:

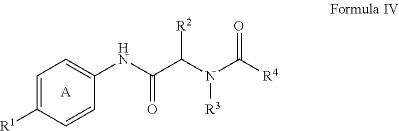

wherein
Ring A is an optionally further substituted 6-membered aromatic ring,
R¹ is
   (1) a group represented by the formula: -Q(R^{1a})(R^{1b})(R^{1c}) wherein
      Q is a carbon atom, a silicon atom or a germanium atom, and
      R^{1a}, R^{1b} and R^{1c} are each independently a substituent, or
      R^{1a} and R^{1b} in combination optionally form, together with the adjacent Q, an optionally substituted ring,
   (2) a neopentyl group, or
   (3) a trimethylsilylmethyl group,
R² is
   (1) a group represented by the formula:

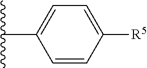

wherein
   R⁵ is an optionally substituted alkyl group or an optionally substituted alkoxy group, and
   the benzene ring in the formula optionally has additional substituent(s) besides R⁵,
(2) an optionally substituted bicyclic fused heterocyclic group, or
(3) a group represented by the formula: -L-Z¹
   wherein
   L is a bond or CH₂, and
   Z¹ is an optionally substituted non-aromatic ring group,
R³ is a hydrogen atom or a substituent, and
R⁴ is a substituent (provided that
   (1) a group represented by the formula:

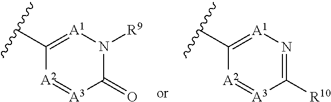

wherein
   A¹ is CR^{A1} wherein R^{A1} is a hydrogen atom or a substituent, or a nitrogen atom,
   A² is CR^{A2} wherein R^{A2} is a hydrogen atom or a substituent, or a nitrogen atom,
   A³ is CR^{A3} wherein R^{A3} is a hydrogen atom or a substituent, or a nitrogen atom, or
   when A² is CR^{A2} wherein R^{A2} is a substituent, and A³ is CR^{A3} wherein R^{A3} is a substituent, then R^{A2} and R^{A3} in combination optionally form, together with the carbon atoms that they are bonded to, a hydrocarbon ring or a heterocycle,
   R⁹ is a hydrogen atom or a hydroxy group, and when R⁹ is a hydroxy group, then A¹, A² and A³ are CR^{A1}, CR^{A2} and CR^{A}, respectively, and
   R¹⁰ is a hydroxy group or an optionally substituted C₁₋₆ alkoxy group, and
(2) an optionally substituted C₁₋₆ alkoxy group are excluded), or when R³ is a substituent, then R³ and R⁴ in combination optionally form, together with the nitrogen atom adjacent to R³ and the carbon atom adjacent to R⁴, an optionally substituted ring (provided that
(1) a cyclic group represented by the formula:

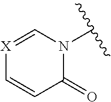

wherein X is CH or a nitrogen atom, which is optionally further substituted, and
(2) a cyclic group represented by the formula:

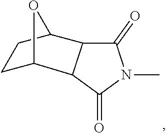

are excluded), and
   the substituents that the ring optionally has optionally form a spiro ring, provided that
5-chloro-N-[1-cyclohexyl-2-oxo-2-[[4-[1-(1-pyrrolidinylmethyl)cyclopropyl]phenyl]amino]ethyl]-2-thiophenecarboxamide,
α-(acetylamino)-N-[4-(trifluoromethyl)phenyl]-cyclopentaneacetamide,
α-(acetylamino)-N-[4-(1,1-dimethylethyl)phenyl]-cyclopentaneacetamide,
α-(acetylamino)-N-[2-bromo-4-(trifluoromethyl)phenyl]-cyclopentaneacetamide, and
N-(4-tert-butyl-2-((5-ethyl-2-(2-ethyl-4,4-dimethylpentyl)-7,7-dimethyloctyl)oxy)phenyl)-2-(5,5-dimethyl-2,4-dioxo-1,3-oxazolidin-3-yl)-2-(2-octadecyl-1,1-dioxido-2H-1,2,4-benzthiadiazin-3-yl)acetamide
   are excluded,

or a stereoisomer, solvates, tautomers, or pharmaceutically acceptable salts thereof.

Further RORγt inhibitors for use in the methods of and compositions described herein are described in US 2017/0107240 A1 and WO 2015/002230 A1, the disclosures of each of which are incorporated herein by reference in their entireties.

In one embodiment, the compounds for use in the presently disclosed compositions and/or methods is a compound of Formula V:

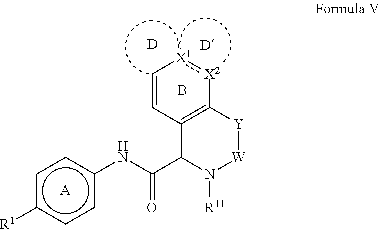

wherein
Ring A is an optionally further substituted 6-membered aromatic ring,
R¹ is
   (1) a group represented by the formula: -Q(R^{1a}) (R^{1b}) (R^{1c}) wherein Q is a carbon atom, a silicon atom or a germanium atom, and Ria, R^{1b} and R^{1c} are each independently a substituent, or R^{1a} and R^{1b} in combination optionally form, together with the adjacent Q, an optionally further substituted ring, and R^{1c} is optionally bonded to one substituent for Ring A to form an optionally further substituted ring,
   (2) a neo-pentyl group, or
   (3) a trimethylsilylmethyl group,
R¹¹ is —CR¹²R^{12′}—R^{12″}, —C(═O)—R⁴ or —SO₂—R¹³,
R¹², R¹² and R^{12′} are each independently a hydrogen atom, a halogen atom, a cyano group, a nitro group, an optionally substituted C₁₋₆ alkyl group, an optionally substituted C₂₋₆ alkenyl group, an optionally substituted C₂₋₆ alkynyl group, an optionally substituted heterocyclic group or an optionally substituted thiocarbamoyl group, R⁴ is an optionally substituted C₁₋₆ alkyl group, an optionally substituted C₂₋₆ alkenyl group, an optionally substituted C₂₋₆ alkynyl group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group or an optionally substituted silyl group, wherein the “C₁₋₆ alkyl group”, the “C₂₋₆ alkenyl group” and the “C₂₋₆ alkynyl group” of the “optionally substituted C₁₋₆ alkyl group”, the “optionally substituted C₂₋₆ alkenyl group” and the “optionally substituted C₂₋₆ alkynyl group” for R⁴ are each optionally substituted by 1 to 5 substituents selected from (1) a halogen atom, (2) a nitro group, (3) a cyano group, (4) an oxo group, (5) a hydroxy group, (6) a C₁₋₆ alkoxy group optionally substituted by substituent(s) selected from a halogen atom and a carboxy group, (7) a C₆₋₁₄ aryloxy group, (8) a C₇₋₁₆ aralkyloxy group, (9) a 5- to 14-membered aromatic heterocyclyloxy group, (10) a 3- to 14-membered non-aromatic heterocyclyloxy group, (11) a C₁₋₆ alkyl-carbonyloxy group, (12) a C₆₋₁₄ aryl-carbonyloxy group, (13) a C₁₋₆ alkoxy-carbonyloxy group, (14) a mono- or di-C₁₋₆ alkyl-carbamoyloxy group, (15) a C₆₋₁₄ aryl-carbamoyloxy group, (16) a 5- to 14-membered aromatic heterocyclylcarbonyloxy group, (17) a 3- to 14-membered non-aromatic heterocyclylcarbonyloxy group, (18) an optionally halogenated C₁₋₆ alkylsulfonyloxy group, (19) a C₆₋₁₄ arylsulfonyloxy group optionally substituted by C₁₋₆ alkyl group(s), (20) an optionally halogenated C₁₋₆ alkylthio group, (21) a 5- to 14-membered aromatic heterocyclic group optionally substituted by substituent(s) selected from a hydroxy group, a C₁₋₆ alkyl group, a C₁₋₆ alkoxy group and a carboxy group, (22) a 3- to 14-membered non-aromatic heterocyclic group optionally substituted by substituent(s) selected from an oxo group and a C₁₋₆ alkyl group, (23) a formyl group, (24) a carboxy group, (25) an optionally halogenated C₁₋₆ alkyl-carbonyl group, (26) a C₆₋₁₄ aryl-carbonyl group, (27) a 5- to 14-membered aromatic heterocyclylcarbonyl group, (28) a 3- to 14-membered non-aromatic heterocyclylcarbonyl group, (29) a C₁₋₆ alkoxy-carbonyl group, (30) a C₆₋₁₄ aryloxy-carbonyl group, (31) a C₇₋₁₆ aralkyloxy-carbonyl group, (32) a carbamoyl group, (33) a thiocarbamoyl group, (34) a mono- or di-C₁₋₆ alkyl-carbamoyl group, (35) a C₆₋₁₄ aryl-carbamoyl group, (36) a 5- to 14-membered aromatic heterocyclylcarbamoyl group, (37) a 3- to 14-membered non-aromatic heterocyclylcarbamoyl group, (38) an optionally halogenated C₁₋₆ alkylsulfonyl group, (39) a C₆₋₁₄ arylsulfonyl group, (40) a 5- to 14-membered aromatic heterocyclylsulfonyl group, (41) an optionally halogenated C₁₋₆ alkylsulfinyl group, (42) a C₆₋₁₄ arylsulfinyl group, (43) a 5- to 14-membered aromatic heterocyclylsulfinyl group, (44) an amino group, (45) a mono- or di-C₁₋₆ alkylamino group (the C₁₋₆ alkyl is optionally substituted by carboxy group(s)), (46) a mono- or di-C₆₋₁₄ arylamino group, (47) a 5- to 14-membered aromatic heterocyclylamino group, (48) a C₇₋₁₆ aralkylamino group, (49) a formylamino group, (50) a C₁₋₆ alkyl-carbonylamino group, (51) a (C₁₋₆ alkyl) (C₁₋₆ alkyl-carbonyl)amino group, (52) a C₆₋₁₄ aryl-carbonylamino group, (53) a C₁₋₆ alkoxy-carbonylamino group, (54) a C₇₋₁₆ aralkyloxy-carbonylamino group, (55) a C₁₋₆ alkylsulfonylamino group, (56) a C₆₋₁₄ arylsulfonylamino group optionally substituted by C₁₋₆ alkyl group(s), (57) an optionally halogenated C₁₋₆ alkyl group, (58) a C₂₋₆ alkenyl group, and (59) a C₂₋₆ alkynyl group,
R¹³ is a substituent,
Ring B is a benzene ring, a pyridine ring or a dihydropyridine ring, each of which is optionally further substituted,
the partial structure represented by the formula:

[symbol]

is CR^{5a}═CR⁶, CR^{5b}═N or C(═O)—NR⁷,
R^{5a} and R^{5b} are each independently an optionally substituted alkyl group, an optionally substituted alkoxy group, an optionally substituted alkylsulfonyl group, a cyano group, an optionally substituted cyclic amino group or an oxetan-3-yloxy group, and
R⁶ and R⁷ are each independently a hydrogen atom or a substituent, or the substituent that Ring B optionally further has and R^{5a} or R^{5b} in combination optionally form Ring D, wherein Ring D is a 5- or 6-membered oxygen-containing heterocycle containing 1 to 2 oxygen atoms as heteroatoms in addition to carbon atoms, and is fused at the ring forming position, or
R^{5a} and R⁶ in combination optionally form Ring D′, wherein Ring D′ is a 5- or 6-membered oxygen-containing heterocycle containing 1 to 2 oxygen atoms as heteroatoms in addition to carbon atoms, and is fused at the ring forming position,
Y is an optionally substituted methylene group or an oxygen atom, and
W is an optionally substituted C₁₋₂ alkylene group,

or a stereoisomer, solvates, tautomers, or pharmaceutically acceptable salts thereof.

In another embodiment, the compounds for use in the presently disclosed compositions and/or methods is a compound of Formula VI:

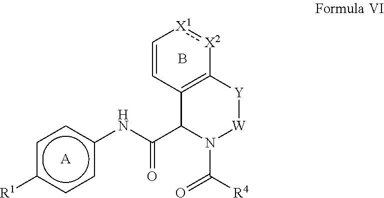

wherein
Ring A is an optionally further substituted 6-membered aromatic ring,
R¹ is
   (1) a group represented by the formula: -Q(R^{1a})(R^{1b}) (R^{1c}) wherein Q is a carbon atom, a silicon atom or a germanium atom, and Ria, R^{1b} and R^{1c} are each independently a substituent, or R^{1a} and R^{1b} in combination optionally form, together with the adjacent Q, an optionally further substituted ring, and R^{1C} is optionally bonded to one substituent for Ring A to form an optionally further substituted ring,
   (2) a neo-pentyl group, or
   (3) a trimethylsilylmethyl group,
R⁴ is a halogen atom, a cyano group, a nitro group, an optionally substituted C₁₋₆ alkyl group, an optionally substituted C₂₋₆ alkenyl group, an optionally substituted C₂₋₆ alkynyl group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group or an optionally substituted silyl group,
wherein the “optionally substituted C₁₋₆ alkyl group”, the “optionally substituted C₂₋₆ alkenyl group” and the “optionally substituted C₂₋₆ alkynyl group” for R⁴ are each optionally substituted by 1 to 5 substituents selected from (1) a halogen atom, (2) a nitro group, (3) a cyano group, (4) an oxo group, (5) a hydroxy group, (6) an optionally halogenated C₁₋₆ alkoxy group, (7) a C₆₋₁₄ aryloxy group, (8) a C₇₋₁₆ aralkyloxy group, (9) a 5- to 14-membered aromatic heterocyclyloxy group, (10) a 3- to 14-membered non-aromatic heterocyclyloxy group, (11) a C₁₋₆ alkyl-carbonyloxy group, (12) a C₆₋₁₄ aryl-carbonyloxy group, (13) a C₁₋₆ alkoxy-carbonyloxy group, (14) a mono- or di-C₁₋₆ alkyl-carbamoyloxy group, (15) a C₆₋₁₄ aryl-carbamoyloxy group, (16) a 5- to 14-membered aromatic heterocyclylcarbonyloxy group, (17) a 3- to 14-membered non-aromatic heterocyclylcarbonyloxy group, (18) an optionally halogenated C₁₋₆ alkylsulfonyloxy group, (19) a C₆₋₁₄ arylsulfonyloxy group optionally substituted by C₁₋₆ alkyl group(s), (20) an optionally halogenated C₁₋₆ alkylthio group, (21) a 5- to 14-membered aromatic heterocyclic group, (22) a 3- to 14-membered non-aromatic heterocyclic group, (23) a formyl group, (24) a carboxy group, (25) an optionally halogenated C₁₋₆ alkyl-carbonyl group, (26) a C₆₋₁₄ aryl-carbonyl group, (27) a 5- to 14-membered aromatic heterocyclylcarbonyl group, (28) a 3- to 14-membered non-aromatic heterocyclylcarbonyl group, (29) a C₁₋₆ alkoxy-carbonyl group, (30) a C₆₋₁₄ aryloxy-carbonyl group, (31) a C₇₋₁₆ aralkyloxy-carbonyl group, (32) a carbamoyl group, (33) a thiocarbamoyl group, (34) a mono- or di-C₁₋₆ alkyl-carbamoyl group, (35) a C₆₋₁₄ aryl-carbamoyl group, (36) a 5- to 14-membered aromatic heterocyclylcarbamoyl group, (37) a 3- to 14-membered non-aromatic heterocyclylcarbamoyl group, (38) an optionally halogenated C₁₋₆ alkylsulfonyl group, (39) a C₆₋₁₄ arylsulfonyl group, (40) a 5- to 14-membered aromatic heterocyclylsulfonyl group, (41) an optionally halogenated C₁₋₆ alkylsulfinyl group, (42) a C₆₋₁₄ arylsulfinyl group, (43) a 5- to 14-membered aromatic heterocyclylsulfinyl group, (44) an amino group, (45) a mono- or di-C₁₋₆ alkylamino group, (46) a mono- or di-C₆₋₁₄ arylamino group, (47) a 5- to 14-membered aromatic heterocyclylamino group, (48) a C₇₋₁₆ aralkylamino group, (49) a formylamino group, (50) a C₁₋₆ alkyl-carbonylamino group, (51) a (C₁₋₆ alkyl) (C₁₋₆ alkyl-carbonyl)amino group, (52) a C₆₋₁₄ aryl-carbonylamino group, (53) a C₁₋₆ alkoxy-carbonylamino group, (54) a C₇₋₁₆ aralkyloxy-carbonylamino group, (55) a C₁₋₆ alkylsulfonylamino group, (56) a C₆₋₁₄ arylsulfonylamino group optionally substituted by C₁₋₆ alkyl group(s), (57) an optionally halogenated C₁₋₆ alkyl group, (58) a C₂₋₆ alkenyl group, and (59) a C₂₋₆ alkynyl group,
Ring B is a benzene ring, a pyridine ring or a dihydropyridine ring, each of which is optionally further substituted,
the partial structure represented by the formula:

[symbol]

is CR^{5a}═CR⁶, CR^{5b}═N or C(═O)—NR⁷,
R^{5a} and R^{5b} are each independently an optionally substituted alkyl group or an optionally substituted alkoxy group,
R⁶ and R⁷ are each independently a hydrogen atom or a substituent,
Y is an optionally substituted methylene group or an oxygen atom, and
W is an optionally substituted C₁₋₂ alkylene group,

or a stereoisomer, solvates, tautomers, or pharmaceutically acceptable salts thereof.

Further RORγt inhibitors for use in the methods of and compositions described herein are described in 10,053,468 B1 and WO 2015/002231 A1, each of which are incorporated herein by reference in their entireties.

In another embodiment, the compounds for use in the presently disclosed compositions and/or methods is a compound of Formula VII:

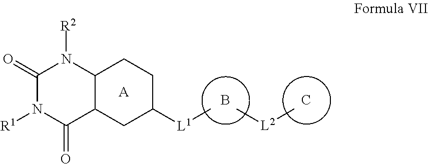

wherein:
R¹ and R² are each independently (1) a methyl group substituted by one substituent selected from (a) an optionally substituted C₃₋₆ cycloalkyl group and (b) an optionally substituted 5- or 6-membered non-aromatic heterocyclic group, (2) an optionally substituted C₂₋₆ alkyl group, or (3) an optionally substituted C₂₋₆ alkenyl group;
ring A is an optionally further substituted 6-membered aromatic ring;
L¹ is a bond, or a spacer having a main chain having 1-3 atoms;
ring B is a non-aromatic ring optionally further substituted by 1 to 3 substituents selected from: (a) an acyl group, (b) an optionally substituted C₁₋₆ alkyl group, (c) an optionally substituted C₁₋₆ alkoxy group, (d) a hydroxy group, (e) a halogen atom and (f) an oxo group;
L² is a bond, or a spacer having a main chain having 1-4 atoms; and
ring C is an optionally further substituted ring,
or a stereoisomer, solvates, tautomers, or pharmaceutically acceptable salts thereof.

In some embodiments, the present disclosure provides a compound according to Formula VII, wherein R¹ and R² are each independently (1) a methyl group substituted by one substituent selected from (a) a C₃₋₆ cycloalkyl group optionally substituted by 1 to 3 halogen atoms and (b) a 5- or 6-membered non-aromatic heterocyclic group, (2) a C₂₋₆ alkyl group optionally substituted by 1 to 3 substituents selected from a halogen atom, a C₁₋₆ alkoxy group and an acyl group, or (3) a C₂₋₆ alkenyl group, or a stereoisomer, solvates, tautomers, or pharmaceutically acceptable salts thereof.

In some embodiments, the present disclosure provides a compound according to Formula VII, wherein L¹ is a bond, or a spacer having a main chain of 1-2 atoms, or a stereoisomer, solvates, tautomers, or pharmaceutically acceptable salts thereof.

In some embodiments, the present disclosure provides a compound according to Formula VII, wherein R² is an optionally substituted C₃₋₆ alkyl group or an optionally substituted C₃₋₆ alkenyl group, each of which is branched at a carbon atom bonded to a nitrogen atom, or a stereoisomer, solvates, tautomers, or pharmaceutically acceptable salts thereof.

In some embodiments, the present disclosure provides a compound according to Formula VII, wherein R¹ is a C₂₋₆ alkyl group optionally substituted by 1 to 3 substituents selected from (1) a methyl group substituted by one substituent selected from (a) a C₃₋₆ cycloalkyl group optionally substituted by 1 to 3 halogen atoms and (b) a 5- or 6-membered non-aromatic heterocyclic group, or (2) a halogen atom, a C₁₋₆ alkoxy group and a C₁₋₆ alkoxy-carbonyl group; R² is (1) a methyl group substituted by a C₃₋₆ cycloalkyl group, (2) a C₂₋₆ alkyl group optionally substituted by 1 to 3 halogen atoms, or (3) a C₂₋₆ alkenyl group; ring A is (1) a benzene ring optionally further substituted by 1 to 3 halogen atoms, or (2) 6-membered aromatic heterocycle; L¹ is a bond, —C(═O)—, —O—C(═O)—, —CH₂—C(═O)—, —C(═O)—NH—, or —NH—C(═O)—; ring B is C₃₋₁₀ cycloalkane or non-aromatic heterocycle, each of which is optionally further substituted by 1 to 3 substituents selected from (a) an acyl group selected from (i) a carboxy group, (ii) a C₁₋₆ alkyl-carbonyl group optionally substituted by a carboxy group, (iii) a C₁₋₆ alkoxy-carbonyl group optionally substituted by a carboxy group or a C₇₋₁₆ aralkyloxy-carbonyl group, (iv) a C₇₋₁₆ aralkyloxy-carbonyl group, (v) a carbamoyl group and (vi) a C₁₋₆ alkyl-sulfonyl group, (b) a C₁₋₆ alkyl group optionally substituted by a hydroxy group, (c) a hydroxy group and (d) an oxo group; L² is a bond, —O—, —C(═O)—, —CH₂O—, —C(═O)—CH₂—, —C(═O)—NH— optionally substituted by a C₁₋₆ alkyl group optionally substituted by 1 to 3 halogen atoms, —NH—C(═O)— optionally substituted by a C₁₋₆ alkyl group optionally substituted by 1 to 3 halogen atoms, —NH—S(═O)₂—, —CH₂—C(═O)—NH—, —CH₂—NH—C(═O)—, —O—C(═O)—NH—, —NH—C(═O)—NH—, —NH—C(═O)—CH₂— optionally substituted by a hydroxy group, —CH₂—NH—CH₂— optionally substituted by a C₁₋₆ alkyl group optionally substituted by 1 to 3 halogen atoms, —NH—C(═O)—CH₂—CH₂— or —CH₂—NH—C(═O)—NH—; and ring C is a C₆₋₁₄ aromatic hydrocarbon ring, a 5- or 6-membered monocyclic aromatic heterocycle, a 8- to 14-membered fused polycyclic aromatic heterocycle, a 3- to 8-membered monocyclic non-aromatic heterocycle or a 9- to 14-membered fused polycyclic non-aromatic heterocycle, each of which is optionally further substituted by 1 to 3 substituents selected from (1) a cyano group, (2) a hydroxy group, (3) an oxo group, (4) a halogen atom, (5) a C₁₋₆ alkyl group optionally substituted by 1 to 3 substituents selected from a cyano group, a hydroxy group, a halogen atom, a C₁₋₆ alkoxy group, an amino group, a C₁₋₆ alkoxy-carbonylamino group, a C₁₋₆ alkyl-carbonylamino group optionally substituted by a halogen atom, a C₂₋₆ alkenyl-carbonylamino group and a C₁₋₆ alkyl-aminocarbonyloxy group, (6) a C₂₋₆ alkenyl group optionally substituted by a C₁₋₆ alkyl-carbonyl group, (7) a C₃₋₆ cycloalkyl group, (8) a C₆₋₁₄ aryl group, (9) a C₁₋₆ alkoxy group optionally substituted by 1 to 3 substituents selected from a halogen atom and a C₁₋₆ alkoxy group, (10) a C₁₋₆ alkyl-carbonyl group, (11) a carboxy group, (12) a C₂₋₆ alkenyl-carbonyl group, (13) a C₁₋₆ alkoxy-carbonyl group, (14) a carbamoyl group, (15) an amino group, (16) a C₁₋₆ alkyl-carbonylamino group optionally substituted by a halogen atom, (17) a C₁₋₆ alkoxy-carbonylamino group, (18) a C₁₋₆ alkyl-sulfonyl group, (19) a C₂₋₆ alkenyl-carbonylamino group optionally substituted by a mono- or di-C₁₋₆ alkylamino group, (20) a C₂₋₆ alkenyl-sulfonylamino group and (21) a 3- to 8-membered monocyclic non-aromatic heterocycle; or a stereoisomer, solvates, tautomers, or pharmaceutically acceptable salts thereof.

In some embodiments, the present disclosure provides a compound according to Formula VII, wherein R¹ is a methyl group substituted by one substituent selected from (a) a C₃₋₆ cycloalkyl group optionally substituted by 1 to 3 halogen atoms and (b) a 5- or 6-membered non-aromatic heterocyclic group; R² is a C₂₋₆ alkyl group; ring A is a benzene ring optionally further substituted by 1 to 3 halogen atoms; L¹ is NH—C(═O)—; ring B is a C₃₋₁₀ cycloalkane or a 3- to 8-membered monocyclic non-aromatic heterocycle; L² is a bond, —C(═O)—NH—, —NH—C(═O)— or —NH—C(═O)—NH—; and ring C is a C₆₋₁₄ aromatic hydrocarbon ring, a 5- or 6-membered monocyclic aromatic heterocycle, a 8- to 14-membered fused polycyclic aromatic heterocycle or a 9- to 14-membered fused polycyclic non-aromatic heterocycle, each of which is optionally further substituted by 1 to 3 substituents selected from (1) a cyano group, (2) an oxo group, (3) a halogen atom, (4) a C₁₋₆ alkyl group optionally substituted by 1 to 3 substituents selected from a C₁₋₆ alkoxy-carbonylamino group and a C₁₋₆ alkyl-aminocarbonyloxy group, (5) a C₁₋₆ alkoxy group and (6) a C₁₋₆ alkoxy-carbonyl group; or a stereoisomer, solvates, tautomers, or pharmaceutically acceptable salts thereof.

Further RORγt inhibitors for use in the methods of and compositions described herein are described in U.S. Pat. No. 10,000,488 B1 and WO 2016/039408 A1, each of which are incorporated herein by reference in their entireties.

In further embodiments, the compounds for use in the presently disclosed compositions and/or methods is selected from one or more of the following:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)-yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindol-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N-(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide;
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide (3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)carbamoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)-yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-2-((4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide;

or a stereoisomer, solvates, tautomers, or pharmaceutically acceptable salts thereof

In a first aspect, the present disclosure provides for a dermatological composition [Composition 1] comprising:
a pharmaceutically effective amount of a RORγt inhibitor (e.g., a RORγt inhibitor of the present disclosure);
a dermatologically acceptable carrier;
a humectant; and
a preservative.

The present disclosure further provides compositions as follows:
1.1 Composition 1, wherein the RORγt inhibitor is a compound according to any of Formulas I, II. III, IV, V, VI, or VII.
1.2 Any of the preceding compositions, wherein the RORγt inhibitor is a compound selected from:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)-yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindol-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N-(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide (2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide;
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)carbamoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)-yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-2-((4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide;
   or a stereoisomer, solvates, tautomers, or pharmaceutically acceptable salts thereof.
   1.3 Any of the preceding compositions, wherein the carrier comprises water (e.g., deionized water), an alcohol (e.g., ethanol, 2-propanol and n-propanol) and/or a glycol (e.g., polyethylene glycol, e.g., PEG 200, PEG 300, PEG 400).
   1.4 Any of the preceding compositions, wherein the carrier comprises one or more of water, ethanol and/or polyethylene glycol (e.g., PEG 300).
   1.5 Any of the preceding compositions, wherein the carrier comprises water and/or ethanol.
   1.6 Any of the preceding compositions, wherein the carrier comprises or consists of water.
   1.7 Any of the preceding compositions, wherein the carrier comprises or consists of polyethylene glycol.
   1.8 Any of the preceding compositions, wherein the carrier is present in an amount of about 25 wt. % to about 75 wt. %, about 40 wt. % to about 70 wt. %, about 45 wt. % to about 60 wt. %, or about 55 wt. % to about 70 wt. %, based on the total weight of the composition.
   1.9 Any of the preceding compositions, wherein the carrier is present in an amount of about 40 wt. %, about 45 wt. %, about 50 wt. %, about 55 wt. %, about 60 wt. %, about 65 wt. %, about 70 wt. %, based on the total weight of the composition.
   1.10 Any of the preceding compositions, wherein the composition contains less than 1% polyethylene glycol (e.g., PEG 400).
   1.11 Any of the preceding compositions, wherein the composition contains less than 0.1% polyethylene glycol (e.g., PEG 400).
   1.12 Any of the preceding compositions, wherein the composition contains less than 0.001% polyethylene glycol (e.g., PEG 400).
   1.13 Any of the preceding compositions, wherein the composition is substantially free from polyethylene glycol (e.g., PEG 400).
   1.14 Any of the preceding compositions, wherein the humectant comprises one or more of a polyhydric alcohol and/or a silicone oil.
   1.15 Any of the preceding compositions, wherein the humectant comprises one or more of glycerol, sorbitol, cyclomethicone polypropylene glycol, and/or propylene glycol.
   1.16 Any of the preceding compositions, wherein the humectant comprises or consists of glycerol, propylene glycol and/or sorbitol.
   1.17 Any of the preceding compositions, wherein the humectant comprises or consists of glycerol and propylene glycol.
   1.18 Any of the preceding compositions, wherein the humectant comprises or consists of glycerol.
   1.19 Any of the preceding compositions, wherein the humectant comprises or consists of propylene glycol.
   1.20 Any of the preceding compositions, wherein the humectant is present in an amount of about 5 wt. % to about 40 wt. %, about 15 wt. % to about 30 wt. %, or about 25 wt. % to about 30 wt. %, e.g., about 5 wt. % to about 10 wt. %, such as about 5 wt. %, about 6 wt. %, about 7 wt. %, about 8 wt. %, about 9 wt. %, or about 10 wt. %, based on the total weight of the composition.
   1.21 Any of the preceding compositions, wherein the humectant comprises glycerol in an amount of about 5 wt. % to about 20 wt. % or about 8 wt. % to about 12 wt. %, e.g., about 5 wt. %, about 10 wt. %, about 15 wt. % or about 20 wt. %, based on the total weight of the composition.
   1.22 Any of the preceding compositions, wherein the humectant comprises propylene glycol in an amount of about 1 wt. % to about 25 wt. %, about 5 wt. % to about 20 wt. %, or about 15 wt. % to about 25 wt. %, e.g., about 5 wt. %, about 10 wt. %, about 15 wt. %, about 16 wt. %, about 17 wt. %, about 18 wt. %, about 19 wt. %, about 20 wt. %, or about 25 wt. %, based on the total weight of the composition.
   1.23 Any of the preceding compositions, wherein the composition contains less than 1% sorbitol.
   1.24 Any of the preceding compositions, wherein the composition contains less than 0.1% sorbitol.
   1.25 Any of the preceding compositions, wherein the composition contains less than 0.001% sorbitol.
   1.26 Any of the preceding compositions, wherein the composition is substantially free from sorbitol.
   1.27 Any of the preceding compositions, wherein the preservative comprises one or more of sodium benzoate, benzyl alcohol, diazolidinyl urea, methylparaben, propylparaben, tetrasodium EDTA, and/or ethylparaben.
   1.28 Any of the preceding compositions, wherein the preservative comprises one or more of sodium benzoate and/or benzyl alcohol.
   1.29 Any of the preceding compositions, wherein the preservative comprises or consists of sodium benzoate in an amount of about 0.01 wt. % to about 1 wt. %, e.g., about 0.2 wt. %, based on the total weight of the composition.
   1.30 Any of the preceding compositions, wherein the preservative comprises or consists of benzyl alcohol in an amount of about 1 wt. % to about 5 wt. %, e.g., about 2 wt. %, based on the total weight of the composition.
   1.31 Any of the preceding compositions, further comprising a skin absorption enhancer.
   1.32 Any of the preceding compositions, comprising a skin absorption enhancer selected from one or more of a C₁₋₂₀ alkanol (e.g., oleyl alcohol, cetyl alcohol, octyldodecanol, cetostearyl alcohol, benzyl alcohol), a saturated or unsaturated fatty acid ester, a saturated or unsaturated fatty acid ester, a polyoxythylene fatty ether, a polyoxylene fatty acid esters, diethylene glycol monoethyl ether, 1,3-dimethyl-2-imidazolidinone and/or dimethyl isosorbide.
   1.33 Any of the preceding compositions, comprising a skin absorption enhancer selected from one or more of oleyl alcohol, cetyl alcohol, octyldodecanol, cetostearyl alcohol, mineral oil, benzyl alcohol, isopropyl myristate, diisopropyl adipate, ethylhexyl hydroxystearate, Steareth-2 (Brij S2), Steareth-20 (Brij S20), glyceryl stearate, stearic acid, magnesium stearate, diethylene glycol monoethyl ether, 1,3-dimethyl-2-imidazolidinone and/or dimethyl isosorbide.
   1.34 Any of the preceding compositions, comprising a skin absorption enhancer in an amount of about 10 wt. % to about 45 wt. %, about 15 wt. % to about 25 wt. %, about 15 wt. % to about 20 wt. %, e.g., about 15 wt. %, about 16 wt. %, about 17 wt. %, about 18 wt. %, about 19 wt. %, or about 20 wt. %, based on the total weight of the composition.
   1.35 Any of the preceding compositions, comprising diethylene glycol monoethyl ether in an amount of about 10 wt. % to about 45 wt. %, about 15 wt. % to about 25 wt. %, about 15 wt. % to about 20 wt. %, e.g., about 15 wt. %, about 16 wt. %, about 17 wt. %, about 18 wt. %, about 19 wt. %, or about 20 wt. %, based on the total weight of the composition.
   1.36 Any of the preceding compositions, wherein the composition comprises propylene glycol in an amount of about 5 wt. % to about 20 wt. % and diethylene glycol monoethyl ether in an amount of about 20 wt. % to about 40 wt. %, based on the total weight of the composition.
   1.37 Any of the preceding compositions, wherein the composition comprises propylene glycol and diethylene glycol monoethyl ether in a total amount of greater than about 30 wt. %.
   1.38 Any of the preceding compositions, wherein the composition comprises propylene glycol and diethylene glycol monoethyl ether in a total amount of about 30 wt. % to about 40 wt. % or about 35 wt. % to about 40 wt. %, e.g., about 35 wt. %, about 35 wt. %, about 36 wt. %, about 37 wt. %, about 38 wt. %, about 39 wt. %, or about 40 wt. %, based on the total weight of the composition.
   1.39 Any of the preceding compositions, further comprising a viscosity enhancing agent.
   1.40 Any of the preceding compositions, comprising a viscosity enhancing agent selected from one or more of a cellulose, an acrylate polymer or crosspolymers, or a carbomer.
   1.41 Any of the preceding compositions, comprising a viscosity enhancing agent selected from hydroxypropyl cellulose, hydroxyethyl cellulose, hydroxymethyl cellulose, hydroxypropylmethyl cellulose (e.g., Benecel E4M), poloxamer (Pluronic PF127), carbomers (e.g., carbomer 980, carbomer 1342 and carbomer 940), more specifically hydroxypropyl cellulose (e.g., hydroxypropyl cellulose having a molecular weight between 850,000-1,150,000 daltons Klucel® EF, GF, MF and/or HF), Pluronic PF127, carbomer 980 and/or carbomer 1342 (Pemulen® TR-1, TR-2 and/or Carbopol® ETD 2020).
   1.42 Any of the preceding compositions, comprising a viscosity enhancing agent in an amount of about 1 wt. % to about 5 wt. % (e.g., about 2 wt. %), based on the total weight of the composition.
   1.43 Any of the preceding compositions, further comprising a chelating agent.
   1.44 Any of the preceding compositions, comprising a chelating agent selected from one or more of EDTA (e.g., disodium EDTA), disodium edeate, dipotassium edeate, cyclodextrin, trisodium edetate, tetrasodium edetate, citric acid, sodium citrate, gluconic acid and potassium gluconate.
   1.45 Any of the preceding compositions, comprising EDTA.
   1.46 Any of the preceding compositions, comprising a chelating agent in an amount of about 0.01 wt. % to about 1 wt. %, e.g., about 0.1 wt. %.
   1.47 Any of the preceding compositions, further comprising an antioxidant.
   1.48 Any of the preceding compositions, comprising an antioxidant selected from one or more of butylated hydroxytoluene (BHT), sodium metabisulfite, ascorbic acid, propyl gallate, and/or alpha tocopherol (Vitamin E).
   1.49 Any of the preceding compositions, comprising an antioxidant in an amount of about 0.001 wt. % to about 1 wt. %, based on the total weight of the composition.
   1.50 Any of the preceding compositions, wherein the antioxidant comprises or consists of butylated hydroxytoluene in an amount of about 0.01 wt. % to about 1 wt. %, e.g., about 0.2 wt. %, based on the total weight of the composition.
   1.51 Any of the preceding compositions, wherein the antioxidant comprises or consists of ascorbic acid in an amount of about 0.01 wt. % to about 1 wt. %, e.g., about 0.1 wt. %, based on the total weight of the composition.
   1.52 Any of the preceding compositions, wherein the antioxidant comprises or consists of propyl gallate in an amount of about 0.01 wt. % to about 0.1 wt. %, e.g., about 0.05 wt. %, based on the total weight of the composition.
   1.53 Any of the preceding compositions, wherein the antioxidant comprises or consists of alpha tocopherol (Vitamin E) in an amount of about 0.0001 wt. % to about 0.01 wt. %, e.g., about 0.002 wt. %, based on the total weight of the composition.
   1.54 Any of the preceding compositions, wherein the antioxidant comprises or consists of sodium metabisulfite in an amount of about 0.01 wt. % to about 1 wt. %, e.g., about 0.1 wt. %, about 0.2 wt. %, about 0.3 wt. %, about 0.4 wt. %, or about 0.5 wt. % based on the total weight of the composition.
   1.55 Any of the preceding compositions, wherein the composition contains less than 1% sodium metabisulfite.
   1.56 Any of the preceding compositions, wherein the composition contains less than 0.1% sodium metabisulfite.
   1.57 Any of the preceding compositions, wherein the composition contains less than 0.001% sodium metabisulfite.
   1.58 Any of the preceding compositions, wherein the composition is substantially free from sodium metabisulfite.
   1.59 Any of the preceding compositions, further comprising a pH adjuster selected from one or more of citric acid, phosphoric acid, and/or an alkali hydroxide (e.g., sodium hydroxide).
   1.60 Any of the preceding compositions, further comprising a surfactant.
   1.61 Any of the preceding compositions, comprising a surfactant selected from one or more of polysorbate (e.g., polysorbate 20), polyethylene glycol hexadecyl ether (Cetomacrogol 1000), and/or a dispersion of dispersion of acrylamide/sodium acryloyldimethyl taurate copolymer/isohexadecane and polysorbate.
   1.62 Any of the preceding compositions, wherein the composition is in the form of a cream, a lotion, a foam, an aqueous gel, a non-aqueous gel, a spray or an ointment (e.g., a polyethylene glycol-based ointment).
   1.63 Any of the preceding compositions, wherein the composition is in the form of an aqueous gel.
   1.64 Any of the preceding compositions, wherein the RORγt inhibitor is present at a concentration of about 0.001 wt. % to about 25 wt. %, based on the total weight of the composition.
   1.65 Any of the preceding compositions, wherein the RORγt inhibitor is present at a concentration of about 0.1 wt. % to about 5 wt. %, based on the total weight of the composition.
   1.66 Any of the preceding compositions, wherein the RORγt inhibitor is present at a concentration of about 2 wt. % to about 3 wt. %, e.g. about 2 wt. % or about 3 wt. %, based on the total weight of the composition.
   1.67 Any of the preceding compositions, wherein the composition has a pH of about 3.5 to about 7.5, about 4 to about 7, about 4.5 to about 6.5 or about 5 to about 6.5.
   1.68 Any of the preceding compositions, wherein the composition is an oil-in-water emulsion or an oil-in-water emulsion.
   1.69 The preceding composition, wherein the composition is applied to a patient's skin once daily, every other day, weekly, or monthly.
   1.70 The preceding composition, wherein the composition is applied to a patient's skin twice daily.
   1.71 The preceding composition, wherein the composition is applied to a patient's skin three times or more daily.
   1.72 Any of the preceding compositions, wherein the composition is administered to a patient suffering from an autoimmune disorder.
   1.73 Any of the preceding compositions, wherein the autoimmune disorder is an autoimmune disorder of the skin.
   1.74 Any of the preceding compositions, wherein the skin is mammalian skin (e.g., human skin).
   1.75 Any of the preceding compositions, wherein the autoimmune disorder is consequent to dysfunction of Th17 cells or the release of IL-17 (e.g., IL-17A).
   1.76 Any of the preceding compositions, wherein the autoimmune disorder is psoriasis, dermatitis, alopecia, cutaneous graft-versus-host-disease (cGVHD), ichthyosis, systemic sclerosis, vitiligo, rosacea, acne, urticaria, Behcet's disease, or lupus erythematosus.
   1.77 Any of the preceding compositions, wherein the autoimmune disorder is psoriasis.
   1.78 Any of the preceding compositions, wherein the autoimmune disorder is psoriasis vulgaris (i.e., plaque psoriasis), guttate psoriasis, inverse psoriasis (i.e., flexural psoriasis), pustular psoriasis (e.g., Von Zumbusch psoriasis, palmoplantar pustulosis, acropustulosis), psoriatic arthritis, erythrodermic psoriasis, nail psoriasis or scalp psoriasis.
   1.79 Any of the preceding compositions, wherein the autoimmune disorder is psoriasis vulgaris (i.e., plaque psoriasis).
   1.80 Any of the preceding compositions, wherein the autoimmune disorder is dermatitis.
   1.81 Any of the preceding compositions, wherein the autoimmune disorder is atopic dermatitis (e.g., mild, moderate, or severe (including mild-to-moderate and moderate-to-severe) atopic dermatitis, Asian atopic dermatitis, European atopic dermatitis, pediatric atopic dermatitis), stasis dermatitis, contact dermatitis, dyshidriotic dermatitis, seborrheic dermatitis, neurodermatitis, nummular dermatitis, or stasis dermatitis.
   1.82 Any of the preceding compositions, wherein the autoimmune disorder is atopic dermatitis (e.g., Asian atopic dermatitis, European atopic dermatitis).
   1.83 Any of the preceding compositions, wherein the autoimmune disorder is Asian atopic dermatitis.
   1.84 Any of the preceding compositions, wherein the autoimmune disorder is alopecia.
   1.85 Any of the preceding compositions, wherein the autoimmune disorder is alopecia areata (e.g., alopecia totalis, alopecia universalis).
   1.86 Any of the preceding compositions, wherein the autoimmune disorder is alopecia areata (e.g., alopecia totalis, alopecia universalis), cicatricial alopecia (e.g., frontal fibrosing alopecia, lichen planus (e.g., lichen planopilaris), folliculitis decalvans), telogen effluvium or anagen effluvium.
   1.87 Any of the preceding compositions, wherein the autoimmune disorder is alopecia areata (e.g., alopecia totalis, alopecia universalis).
   1.88 Any of the preceding compositions, wherein the autoimmune disorder is ichthyosis.
   1.89 Any of the preceding compositions, wherein the autoimmune disorder is acquired ichthyosis, autosomal ichthyosis, autosomal recessive congenital ichthyosis (e.g. CIE or lamellar), Chanarin-Dorfman syndrome, CHILD syndrome, chondrodysplasia punctata syndrome, Darier disease, epidermal nevus syndrome, epidermolytic ichthyosis, erythrokeratodermias, Giroux-Barbeau syndrome, harlequin ichthyosis, Hailey-Hailey disease, ichthyosis en confetti, ichthyosis hystrix, ichthyosis vulgaris, keratosis follicularis spinulosa decalvans, keratitis-ichthyosis-deafness syndrome, multiple sulfatase deficiency, Netherton syndrome, pachyonychia congenita, palmoplantar keratoderma, peeling skin syndrome, pityriasis rubra pilaris, Refsum disease, Rud's syndrome, Sjögren-Larsson syndrome, trichiothiodystrophy, or X-linked ichthyosis.
   1.90 Any of the preceding compositions, wherein the autoimmune disorder is systemic sclerosis.
   1.91 Any of the preceding compositions, wherein the autoimmune disorder is morphea, linear scleroderma, limited systemic scleroderma, diffuse systemic scleroderma, CREST syndrome, sine sclerosis.
   1.92 Any of the preceding compositions, wherein the autoimmune disorder is vitiligo.
   1.93 Any of the preceding compositions, wherein the autoimmune disorder is vitiligo (e.g., non-segmental vitiligo or segmental vitiligo).
   1.94 Any of the preceding compositions, wherein the autoimmune disorder is rosacea.
   1.95 Any of the preceding compositions, wherein the autoimmune disorder is erythematotelangiectatic rosacea, papulopustular rosacea (e.g. acne), rhinophyma, or ocular rosacea.
   1.96 Any of the preceding compositions, wherein the autoimmune disorder is uticaria.
   1.97 Any of the preceding compositions, wherein the autoimmune disorder is chronic spontaneous uticaria.
   1.98 Any of the preceding compositions, wherein the autoimmune disorder is Behcet's disease.
   1.99 Any of the preceding compositions, wherein the autoimmune disorder is lupus erythematosus.
   1.100 Any of the preceding compositions, wherein the autoimmune disorder is systemic lupus erythematosus, discoid lupus erythematosus, or drug-induced lupus.
   1.101 Any of the preceding compositions, wherein the autoimmune disorder is cutaneous graft-versus-host-disease (cGVHD).

As used herein, “topical composition” refers to a formulation of a compound of the invention and a medium generally accepted in the art for the delivery of the biologically active compound to mammalian skin, e.g., human skin. Such a medium includes all dermatologically acceptable carriers, diluents or excipients therefor.

“Stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

“Solvate” refers to a form of a compound complexed by solvent molecules.

“Tautomers” refers to two molecules that are structural isomers that readily interconvert.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

“Dermatologically acceptable excipient” includes without limitation any adjuvant, carrier, vehicle, excipient, glidant, sweetening agent, diluent, preservative, dye/colorant, flavor enhancer, surfactant, wetting agent, dispersing agent, suspending agent, stabilizer, isotonic agent, solvent, or emulsifier, including those approved by the United States Food and Drug Administration as being acceptable for dermatological use on humans or domestic animals, or which are known, or are suitable for use in dermatological compositions.

“Optional” or “optionally” means that the subsequently described event of circumstances may or may not occur, and that the description includes instances where said event or circumstance occurs and instances in which it does not. When a functional group is described as “optionally substituted,” and in turn, substituents on the functional group are also “optionally substituted” and so on, for the purposes of this invention, such iterations are limited to five, preferably such iterations are limited to two.

### Methods of Using the Compounds of the Invention

The Compounds of the Invention are useful in the treatment of autoimmune disorders, e.g., psoriasis. Therefore, administration or use of a preferred RORγt inhibitor as described herein, e.g., a RORγt inhibitor as hereinbefore described, e.g., a Compound of Formula I, II, III, IV, VI, or VII provides a means to regulate the polarization of Th17 cells and the release of inflammatory cytokines (e.g., IL-17A), and in certain embodiments provide a treatment for various autoimmune diseases and disorders.

For example, in one embodiment the present disclosure provides for a method [Method 1] for treating an autoimmune disorder, the method comprising topically administering to a subject in need thereof a topical composition having a therapeutically effective amount of a RORγt inhibitor (e.g. a RORγt inhibitor according to the present disclosure).

The present disclosure further provides further embodiments of Method 1 as follows
1.1 Method 1, wherein the topical composition is a composition according to any of Compositions 1, et. seq.
1.2 Any of the preceding methods, wherein the composition is applied to a patient's skin once daily.
1.3 Any of the preceding methods, wherein the composition is applied to a patient's skin twice daily.
1.4 Any of the preceding methods, wherein the composition is applied to a patient's skin three times or more daily.
1.5 Any of the preceding methods, wherein the autoimmune disorder is an autoimmune disorder of the skin.
1.6 The preceding method, wherein the skin is mammalian skin (e.g., human skin).
1.7 Any of the preceding methods, wherein the autoimmune disorder is consequent to dysfunction of Th17 cells or the release of IL-17 (e.g., IL-17A).
1.8 Any of the preceding methods, wherein the autoimmune disorder is psoriasis, dermatitis, alopecia, cutaneous graft-versus-host-disease (cGVHD), ichthyosis, systemic sclerosis, vitiligo, rosacea, acne, urticaria, Behcet's disease, or lupus erythematosus.
1.9 Any of the preceding methods, wherein the autoimmune disorder is psoriasis.
1.10 Any of the preceding methods, wherein the autoimmune disorder is psoriasis vulgaris (i.e., plaque psoriasis), guttate psoriasis, inverse psoriasis (i.e., flexural psoriasis), pustular psoriasis (e.g., Von Zumbusch psoriasis, palmoplantar pustulosis, acropustulosis), psoriatic arthritis, erythrodermic psoriasis, nail psoriasis or scalp psoriasis.
1.11 Any of the preceding methods, wherein the autoimmune disorder is psoriasis vulgaris (i.e., plaque psoriasis).
1.12 Any of the preceding methods, wherein the autoimmune disorder is dermatitis.
1.13 Any of the preceding methods, autoimmune disorder is atopic dermatitis (e.g., mild, moderate, or severe (including mild-to-moderate and moderate-to-severe) atopic dermatitis, Asian atopic dermatitis, European atopic dermatitis, pediatric atopic dermatitis), stasis dermatitis, contact dermatitis, dyshidriotic dermatitis, seborrheic dermatitis, neurodermatitis, nummular dermatitis, or stasis dermatitis.
1.14 Any of the preceding methods, wherein the autoimmune disorder is atopic dermatitis (e.g., Asian atopic dermatitis, European atopic dermatitis).
1.15 Any of the preceding methods, wherein the autoimmune disorder is Asian atopic dermatitis.
1.16 Any of the preceding methods, wherein the autoimmune disorder is alopecia.
1.17 Any of the preceding methods, wherein the autoimmune disorder is alopecia areata (e.g., alopecia totalis, alopecia universalis), cicatricial alopecia (e.g., frontal fibrosing alopecia, lichen planus (e.g., lichen planopilaris), folliculitis decalvans), telogen effluvium or anagen effluvium.
1.18 Any of the preceding methods, wherein the autoimmune disorder is alopecia areata (e.g., alopecia totalis, alopecia universalis).
1.19 Any of the preceding methods, wherein the autoimmune disorder is ichthyosis.
1.20 Any of the preceding methods, wherein the autoimmune disorder is acquired ichthyosis, autosomal ichthyosis, autosomal recessive congenital ichthyosis (e.g. CIE or lamellar), Chanarin-Dorfman syndrome, CHILD syndrome, chondrodysplasia punctata syndrome, Darier disease, epidermal nevus syndrome, epidermolytic ichthyosis, erythrokeratodermias, Giroux-Barbeau syndrome, harlequin ichthyosis, Hailey-Hailey disease, ichthyosis en confetti, ichthyosis hystrix, ichthyosis vulgaris, keratosis follicularis spinulosa decalvans, keratitis-ichthyosis-deafness syndrome, multiple sulfatase deficiency, netherton syndrome, pachyonychia congenita, palmoplantar keratoderma, peeling skin syndrome, pityriasis rubra pilaris, Refsum disease, Rud's syndrome, Sjögren-Larsson syndrome, trichiothiodystrophy, or X-linked ichthyosis.
1.21 Any of the preceding methods, wherein the autoimmune disorder is systemic sclerosis.
1.22 Any of the preceding methods, wherein the autoimmune disorder is morphea, linear scleroderma, limited systemic scleroderma, diffuse systemic scleroderma, CREST syndrome, sine sclerosis.
1.23 Any of the preceding methods, wherein the autoimmune disorder is vitiligo.
1.24 Any of the preceding methods, wherein the autoimmune disorder is vitiligo (e.g., non-segmental vitiligo or segmental vitiligo).
1.25 Any of the preceding methods, wherein the autoimmune disorder is rosacea.
1.26 Any of the preceding methods, wherein the autoimmune disorder is erythematotelangiectatic rosacea, papulopustular rosacea (e.g. acne), rhinophyma, or ocular rosacea.
1.27 Any of the preceding methods, wherein the autoimmune disorder is uticaria.
1.28 Any of the preceding methods, wherein the autoimmune disorder is chronic spontaneous uticaria.
1.29 Any of the preceding methods, wherein the autoimmune disorder is Behcet's disease.
1.30 Any of the preceding methods, wherein the autoimmune disorder is lupus erythematosus.
1.31 Any of the preceding methods, wherein the autoimmune disorder is systemic lupus erythematosus, discoid lupus erythematosus, or drug-induced lupus.
1.32 Any of the preceding methods, wherein the autoimmune disorder is cutaneous graft-versus-host-disease (cGVHD).
1.33 Any of the preceding methods, the subject is a human.
1.34 Any of the preceding methods, wherein the mammalian skin is human skin.

As used herein, “autoimmune disorder” refers to disorders involving the dysregulation of one or more types of T helper cells, e.g., Th17 cells. Autoimmune disorder encompasses various disorders relating to skin inflammation including, for example, psoriasis, atopic dermatitis, and alopecia. Skin inflammation is typically characterized by redness/flushing, pain, pustules, sensation of heat, and/or swelling. The term autoimmune disorder encompasses autoinflammatory disorders, particularly autoinflammatory disorders of the skin.

“Atopic dermatitis” refers to a skin condition involving chronic inflammation, and symptoms of atopic dermatitis include a red, itchy rash. Atopic dermatitis may be present on the skin of any part of the body, but is common on the hands, feet, upper chest, and in the bends of elbows or knees. Additional symptoms of atopic dermatitis may include small raised bumps or thickened, scaly skin.

“Psoriasis” is a chronic skin condition related to an overactive immune response. Psoriasis may be present on may be present on the skin of any part of the body. Symptoms of psoriasis include local inflammation, skin flaking, and thick white or red patches of skin.

“Alopecia” is an autoimmune skin disease, causing hair loss on the scalp, face and sometimes on other areas of the body. In alopecia areata, for example, T cell lymphocytes cluster around affected follicles, causing inflammation and subsequent hair loss.

“Mammal” or “mammalian” includes humans and both domestic animals such as laboratory animals and household pets, (e.g., cats, dogs, swine, cattle, sheep, goats, horses, rabbits), and non-domestic animals such as wildlife and the like.

“Therapeutically effective amount” refers to that amount of a compound of the invention which, when administered to a mammal, preferably a human, is sufficient to effect treatment of the disease or condition of interest in a mammal, preferably a human, having the disease or condition. The amount of a compound of the invention which constitutes a “therapeutically effective amount” will vary depending on the compound, the disease or condition and its severity, the manner of administration, and the age of the mammal to be treated, but can be determined routinely by one of ordinary skill in the art having regard to his own knowledge and to this disclosure. Preferably, for purposes of this invention, a “therapeutically effective amount” is that amount of a compound of invention which is sufficient to inhibit inflammation of the skin.

“Treating” or “treatment”, as used herein, covers the treatment of the disease or condition of interest in a mammal, preferably a human, and includes:
(i) preventing the disease or condition from occurring in the mammal;
(ii) inhibiting the disease or condition in the mammal, i.e., arresting its development;
(iii) relieving the disease or condition in the mammal, i.e., causing regression of the disease or condition; or
(iv) relieving the symptoms of the disease or condition in the mammal, i.e., relieving the symptoms without addressing the underlying disease or condition.

As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

In the present description, the term “about” means ±20% of the indicated range, value, or structure, unless otherwise indicated.

In some embodiments, the RORγt inhibitor (e.g. a RORγt inhibitor according to the present disclosure) is present in the topical composition at a concentration of about 0.001% to about 50% by weight, e.g., a concentration of about 0.01% to about 30% by weight, a concentration of about 0.1% to about 25% by weight, a concentration of about 0.1% to about 20% by weight, or a concentration of about 1% to about 15% by weight, e.g., a concentration of about 1%, 2%, 3%, 4%, 5%, 6%, 7%, 8%, 9%, 10%, 11%, 12%, etc.

In some embodiments, a therapeutically effective dosage should be from about 0.0001 mg to about 1000 mg per day. In some embodiments, a therapeutically effective dosage can be from about 0.001-50 mg of active ingredient (Compound of Formula I as described herein) per kilogram of body weight per day, delivered topically as descried herein. In some embodiments, the Compound of Formula I is administered at a dosage of up to 1500 mg/day, for example 1200 mg/day, 900 mg/day, 850 mg/day, 800 mg/day, 750 mg/day, 700 mg/day, 650 mg/day, 600 mg/day, 550 mg/day, 500 mg/day, 450 mg/day, 400 mg/day, 350 mg/day, 300 mg/day, 250 mg/day, 200 mg/day, 150 mg/day, 1000 mg/day, 50 mg/day, 25 mg/day, 10 mg/day, or 9, 8, 7, 6, 5, 4, 3, 2, 1, 0.75, 0.5, 0.25, 0.10, 0.05 or 0.01 mg/day.

A dermatological composition of the present disclosure can be in any form useful for topical administration, including a solution, lotion, foam, gel, cream and/or ointment.

In certain embodiments, the pharmaceutical compositions described herein further include a dermatologically acceptable excipient. The dermatologically acceptable excipients may be one or more solvents that solubilize and/or stabilize the active ingredient (e.g., the compound of Formula I) contained therein. The dermatologically acceptable excipients may also include skin absorption enhancers (i.e., penetration enhancers), preservatives, viscosity enhancers, pH adjusters, film forming agents and the like. Non-limiting examples of the suitable excipients include water, polyethylene glycol (e.g., PEG200, PEG300, PEG 400), ethanol, glycerol, Transcutol P (diethylene glycol monoethyl ether), propylene glycol, 1,3-dimethyl-2-imidazolidinone (DMI), sodium metabisulfite, butylated hydroxytoluene (BHT), benzyl alcohol, sodium benzoate, isopropyl myristate, diisopropyl adipate, glyceryl stearate, crodamol OHS (ethylhexyl hydroxystearate), mineral oil, Betadex, polysorbate (Tween 20), steareth-2 (polyoxyethylene (20) stearyl ether; trade name—Brij S2), Steareth-20 (polyoxyethylene (20) stearyl ether; trade name—Brij S20), and/or dimethyl isosorbide (Arlasolve).

More detailed description of certain suitable excipients is described below. As will be appreciated, components of the pharmaceutical formulations described herein can possess multiple functions. For example, a given substance may act as both a viscosity increasing agent and as an emulsifying agent.

As is known, the skin (especially stratum corneum) provides a physical barrier to the harmful effects of the external environment. In doing so, it also interferes with the absorption or transdermal delivery of topical therapeutic drugs. Thus, a suitable dermatologically acceptable excipient may include one or more skin absorption enhancers (or permeation enhancers), which are substances that promote the diffusion of the therapeutic drugs (e.g., the RORγt inhibitors described herein) through the skin barrier. They typically act to reduce the impedance or resistance of the skin to allow improved permeation of the therapeutic drugs. In particular, substances which would perturb the normal structure of the stratum corneum are capable of disrupting the intercellular lipid organization, thus reducing its effectiveness as a barrier. These substances could include any lipid material which would partition into the stratum corneum lipids causing a direct effect or any material which would affect the proteins and cause an indirect perturbation of the lipid structure. Furthermore, solvents, such as ethanol, can remove lipids from the stratum corneum, thus destroying its lipid organization and disrupting its barrier function.

Examples of skin absorption enhancers or barrier function disrupters include, but are not limited to, alcohol-based enhancers, such as alkanols with one to sixteen carbons, such as oleyl alcohol, cetyl alcohol, octyldodecanol, cetostearyl alcohol, benzyl alcohol, butylene glycol, diethylene glycol, glycofurol, glycerides, glycerin, glycerol, phenethyl alcohol, polypropylene glycol, polyvinyl alcohol, and phenol; amide-based enhancers, such as N-butyl-N-dodecylacetamide, crotamiton, N,N-dimethylformamide, N,N-dimethylacetamide, N-methyl formamide, and urea; amino acids, such as L-α-amino acids and water soluble proteins; azone and azone-like compounds, such as azacycloalkanes; essential oils, such as almond oil, amyl butyrate, apricot kernel oil, avocado oil, camphor, castor oil, 1-carvone, coconut oil, corn oil, cotton seed oil, eugenol, menthol, oil of anise, oil of clove, orange oil, peanut oil, peppermint oil, rose oil, safflower oil, sesame oil, shark liver oil (squalene), soybean oil, sunflower oil, and walnut oil; vitamins and herbs, such as aloe, allantoin, black walnut extract, chamomile extract, panthenol, papain, tocopherol, and vitamin A palmitate; waxes, such as candelilla wax, carnauba wax, ceresin wax, beeswax, lanolin wax, jojoba oil, petrolatum; mixes, such as primary esters of fractionated vegetable oil fatty acids with glycerine or propylene glycol, and interesterified medium chain triglyceride oils; saturated or unsaturated fatty acids and related fatty acid esters, such as stearic acid, magnesium stearate, isopropyl myristate, diisopropyl adipate, ethylhexyl hydroxystearate, amyl caproate, butyl acetate, caprylic acid, cetyl ester, diethyl sebacate, dioctyl malate, elaidic acid ethyl caprylate, ethyl glycol palmitostearate, glyceryl stearate, glyceryl beheate, glucose glutamate, isobutyl acetate, laureth-4, lauric acid, malic acid, methyl caprate, mineral oil, myristic acid, oleic acid, palmitic acid, PEG fatty esters, polyoxylene sorbitan monooleate, polypropylene glycols, propylene glycols, saccharose disterate, salicylic acid, sodium citrate, stearic acid, soaps, and caproic-, caprylic-, capric-, and lauric-triglycerides; macrocylics, such as butylated hydroxyanisole, cyclopentadecanolide, cyclodextrins; phospholipid and phosphate enhancers, such as dialkylphosphates, ditetradecyl phosphate, lecithin, 2-pyrrolidone derivatives, such as alkyl pyrrolidone-5-carboxylate esters, pyroglutamic acid esters, N-methyl pyrrolidone, biodegradable soft skin absorption enhancers, such as dioxane derivatives and dioxolane derivatives; sulphoxide enhancers, such as dimethyl sulphoxide and decylmethyl sulphoxide; acid enhancers, such as alginic acid, sorbic acid, and succinic acid; cyclic amines; imidazolinones; imidazoles; ketones, such as acetone, dimethicone, methyl ethyl ketone, and pentanedione; lanolin derivatives, such as lanolin alcohol, PEG 16 lanolin, and acetylated lanolin; oxazolines; oxazolindinones; proline esters; pyrroles, urethanes; polyoxythylene fatty ethers, such as steareth-2 (polyoxyethylene (20) stearyl ether; trade name—Brij S2), Steareth-20 (polyoxyethylene (20) stearyl ether; trade name—Brij S20); and surfactants, such as nonoxynols, polysorbates, polyoxylene alcohols, polyoxylene fatty acid esters, sodium lauryl sulfate, sodium laureth sulfate, polyethylene glycol hexadecyl ether (e.g., including Cetomacrogol 1000), sorbitan monostearate and dispersions of dispersion of acrylamide/sodium acryloyldimethyl taurate copolymer/Isohexadecane (Sepineo P 600).

A dermatological composition of the present disclosure can contain one or more lipophilic solvent(s) and/or hydrophilic co-solvents, that act as a carrier into the pilosebaceous unit. Such solvents can be miscible with water and/or lower chain alcohols and have a vapor pressure less than water at 25° C. (about 23.8 mm Hg). A solvent useful in the compositions of the present disclosure can be a glycol, specifically propylene glycol. In particular, the propylene glycol can be from the class of polyethylene glycols, specifically polyethylene glycols ranging in molecular weight from 200 to 20000. Preferably, the solvent would be part of a class of glycol ethers. More specifically, a solvent of the compositions of the present disclosure would be diethylene glycol monoethyl ether (transcutol). As used herein, “diethylene glycol monoethyl ether” (“DGME”) or “transcutol” refers to 2-(2-ethoxyethoxy)ethanol (CAS NO 001893) or ethyoxydiglycol. Other suitable co-solvents include 1,3-dimethyl-2-imidazolidinone and dimethyl isosorbide.

The topical compositions described herein can contain one or more carriers, which preferably have a vapor pressure greater than or equal to 23.8 mm Hg at 25° C. Preferred concentration range of a single carrier or the total of a combination of carriers can be from about 0.1 wt. % to about 10 wt. %, more preferably from about 10 wt. % to about 50 wt. %, more specifically from about 50 wt. % to about 95 wt. % of the dermatological composition. Non-limiting examples of the solvent include water (e.g., deionized water) and lower alcohols, including ethanol, 2-propanol and n-propanol. More preferably, the carrier is water, ethanol and/or 2-propanol. In some embodiments, the carrier can be ethanol and/or water.

A dermatological composition of the present disclosure can also contain one or more “humectant(s)” used to provide a moistening effect. Preferably the humectant remains stable in the composition. Any suitable concentration of a single humectant or a combination of humectants can be employed, provided that the resulting concentration provides the desired moistening effect. Typically, the suitable amount of humectant will depend upon the specific humectant or humectants employed. Preferred concentration range of a single humectant or the total of a combination of humectants can be from about 0.1 wt. % to about 70 wt. %, more preferably from about 5.0 wt. % to about 30 wt. %, more specifically from about 10 wt. % to about 25 wt. % of the dermatological composition. Non-limiting examples for use herein include glycerin, polyhydric alcohols and silicone oils. More preferably, the humectant is glycerin, polypropylene glycol, propylene glycol, sorbitol and/or cyclomethicone. In some embodiments, the filler can be glycerin and/or sorbitol.

In certain embodiments, the pharmaceutical compositions include a viscosity enhancing agent. The viscosity increasing agent can also act as an emulsifying agent. Typically, the concentration and combination of viscosity increasing agents will depend on the physical stability of the finished product. Preferred concentration range of a viscosity increasing agent can be from about 0.01 wt. % to about 20 wt. %, more preferably from about 0.1 wt. % to about 10 wt. %, more specifically from about 0.5 wt. % to about 5 wt. % of the dermatological composition. Non-limiting examples of viscosity increasing agents for use herein include classes of celluloses, acrylate polymers and acrylate crosspolymers, such as, hydroxypropyl cellulose, hydroxymethyl cellulose, hydroxypropylmethyl cellulose (e.g., Benecel E4M), Pluronic PF127 polymer, carbomers (e.g., carbomer 980, carbomer 1342 and carbomer 940), more specifically hydroxypropyl cellulose (e.g., hydroxypropyl cellulose having a molecular weight between 850,000-1,150,000 daltons Klucel® EF, GF, MF and/or HF), Pluronic PF127, carbomer 980 and/or carbomer 1342 (Pemulen® TR-1, TR-2 and/or Carbopol® ETD 2020). Examples of emulsifiers for use herein include polysorbates, laureth-4, and potassium cetyl sulfate.

A dermatological composition of the present disclosure can contain one or more antioxidants, radical scavengers, and/or stabilizing agents, preferred concentration range from about 0.001 wt. % to about 0.1 wt. %, more preferably from about 0.1 wt. % to about 5 wt. % of the dermatological composition. Examples of suitable antioxidants include, but are not limited to, amino acids such as glycine, histidine, tyrosine, tryptophan and derivatives thereof, imidazoles such as urocanic acid and derivatives thereof, peptides, such as D,L-carnosine, D-carnosine, L-carnosine and derivatives thereof such as anserine, carotenoids, carotenes such as α-carotone, β-carotene, lycopene, and derivatives thereof, chlorogenic acid and derivatives thereof, lipoic acid and derivatives thereof such as dihydrlipoic acid, aurothioglycose, propylthiouracil and other thiols such as thioredoxin, glutathione, cysteine, cystine, cystamine and glycosyl, N-acetyl, methyl, ethyl, propyl, amyl, butyl, lauryl, palmitoyl, oleyl, α-linoleyl, cholesteryl and glyceryl esters and salts thereof, dilauryl thiodipropionate, distearyl thiodipropionate, thiodipropionic acid and derivatives thereof such as esters, ethers, peptides, lipids, nucleotides, nucleosides, and salts, sulfoximine compounds such as buthionine sulfoximines, homocysteine sulfoximine, buthionine sulfones, penta-, hexa-, hepta-thionine sulfoximine, unsaturated fatty acids and derivatives thereof such as α-linolenic acid, linoleic acid, oleic acid, folic acid and derivatives thereof, ubiquinone and ubiquinol and derivatives thereof, vitamin C and derivatives thereof such as ascorbyl palmitate, magnesium ascorbyl phosphate, ascorbyl acetate, tocopherals and derivatives such as vitamin E acetate, vitamin A and derivatives such as vitamin A palmitate, vitamin B and derivatives thereof, coniferyl benzoate of benzoin resin, rutinic acid and derivatives thereof, α-glycosylrutin, ferulic acid, furfurylidene glucitol, carnosine, butyl hydroxytoluene, trihydroxy-butyrophenone, uric acid and derivatives thereof, mannose and derivatives thereof, superoxide dismutase, zinc and derivatives thereof such as zinc oxide, zinc sulfate, selenium and derivatives thereof such as selenium methionine, stilbene and derivatives thereof such as stilbene oxide, trans-stilbene oxide and the like. In particular exemplary embodiments, the one or more antioxidants may include vitamin B, nordihydroguaiaretic acid, butylated hydroxyanisole (BHA), butylated hydroxytoluene (BHT), propyl gallate, erythorbate acid, sodium erythorbate, ascorbir palmitate, and ascorbir stearate. butyl hydroxyanisole, and gallic esters, and in some embodiments, the one or more antioxidants may include BHT. In some embodiments, the antioxidant is selected from one or more of include butylated hydroxytoluene, sodium metabisulfite, butylated hydroxyanisole, ascorbyl palmitate, citric acid, vitamin E, vitamin E acetate, vitamin E-TPGS, ascorbic acid, tocophersolan and propyl gallate. More specifically the anti-oxidant can be metabisulfite, butylated hydroxyanisole, vitamin E, ascorbic acid and/or propyl gallate.

A dermatological composition of the present disclosure can also contain preservatives that exhibit anti-bacterial and/or anti-fungal properties. Preservatives can be present in a gelled dermatological composition of the present disclosure to minimize bacterial and/or fungal over its shelf-life. Preferred concentration range of preservatives in a dermatological composition of the present disclosure can be from about 0.001 wt. % to about 0.01 wt. %, more preferably from about 0.01 wt. % to about 0.5 wt. % of the dermatological composition. Non-limiting examples for use herein include sodium benzoate, sodium benzoate, diazolidinyl urea, methylparaben, propylparaben, tetrasodium EDTA, and ethylparaben. More specifically the preservative would be a combination of methylparaben and propylparaben.

A dermatological composition of the present disclosure can optionally include one or more chelating agents. As used herein, the term “chelating agent” or “chelator” refers to those skin benefit agents capable of removing a metal ion from a system by forming a complex so that the metal ion cannot readily participate in or catalyze chemical reactions. The chelating agents for use herein are preferably formulated at concentrations ranging from about 0.001 wt. % to about 10 wt. %, more preferably from about 0.05 wt. % to about 5.0 wt. % of the dermatological composition. Non-limiting examples for use herein include EDTA (e.g., disodium EDTA), disodium edeate, dipotassium edeate, cyclodextrin, trisodium edetate, tetrasodium edetate, citric acid, sodium citrate, gluconic acid and potassium gluconate. Specifically, the chelating agent can be EDTA, disodium edeate, dipotassium edate, trisodium edetate or potassium gluconate.

The dermatological composition of the present disclosure may be of neutral to mildly acidic pH to allow for comfortable application to the subject's skin, particularly in light of the disease state or condition suffered by the subject. For example, in various embodiments, the pH of the creams may be from about 2.5 to about 7.0, preferably from about 4.0 to about 7.0, more preferably from about 5.0 to about 6.5 at room temperature. In other embodiments, the pH of such creams may be about 5.0, 5.1, 5.2, 5.3, 5.4, 5.5, 5.6, 5.7, 5.8, 5.9, 6.0, 6.1, 6.2, 6.3, 6.4 or 6.5 at room temperature. Any components or combination of components known and useful in the art may be used to achieve an appropriate pH such as, for example, pH adjusters including, but not limited to, lactic acid, citric acid, sodium citrate, glycolic acid, succinic acid, phosphoric acid, monosodium phosphate, disodium phosphate, oxalic acid, dl-malic acid, calcium carbonate, sodium hydroxide, magnesium hydroxide, sodium carbonate, sodium hydrogen carbonate, and ammonium hydrogen carbonate. In certain embodiments the pH regulators comprise a citrate buffer or a phosphate buffer. In some embodiments, the pH adjuster comprises an alkali or alkaline earth hydroxide, e.g. sodium hydroxide or magnesium hydroxide. In various embodiments, the total buffer capacity may be from about from about 0 mM to about 600 mM; from about 0 mM to about 600 mM; from about 5 mM to about 600 mM; from about 5 mM to about 400 mM; from about 5 mM to about 300 mM; from about 5 mM to about 200 mM; from about 200 mM to about 400 mM; about 0 mM, about 100 mM, about 200 mM, about 300 mM, about 400 mM, about 500 mM, or about 600 mM. In some embodiments the cream comprises each pH regulator in an amount of about 0.05%, about 0.1%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.31%, about 0.32%, about 0.33%, about 0.34%, about 0.35%, about 0.36%, about 0.37%, about 0.38%, about 0.39%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% by weight.

The topical compositions described herein may be provided in any cosmetically suitable form, preferably as a lotion, a cream, or a ointment, as well as a sprayable liquid form (e.g., a spray that includes the RORγt inhibitor in a base, vehicle or carrier that dries in a cosmetically acceptable way without the greasy appearance that a lotion or ointment would have when applied to the skin).

Any suitable amount of a RORγt inhibitor (e.g., a compound according to the present disclosure) can be employed in such dermatological compositions, provided the amount effectively reduces local inflammation, and remains stable in the composition over a prolonged period of time. Preferably, the stability is over a prolonged period of time, e.g., up to about 3 years, up to 1 year, or up to about 6 months, which is typical in the manufacturing, packaging, shipping and/or storage of dermatologically acceptable compositions. A compound of the present disclosure can be in solution, partially in solution with an undissolved portion or completely undissolved suspension. A compound of the present disclosure can be present in a dermatological composition of the invention in a concentration range from about 0.001 wt. % to about 80 wt. %, from about 0.001 wt. % to about 50 wt. %, from about 0.001 wt. % to about 25 wt. %, or from about 0.001 wt. % to about 6 wt. % of the dermatological composition. In one embodiment, a compound of the present disclosure can be present in a concentration range of from about 0.001 wt. % to about 10 wt. %, from about 0.1 wt. % to about 10 wt. % or from about 1.0 wt. % to about 5.0 wt. % of the dermatological composition.

In treating the autoimmune disorders, e.g., psoriasis, alopecia, or atopic dermatitis, the topical composition comprising a compound of the present disclosure is preferably administered directly to the affected area of the skin (e.g., psoriasis lesion) of the human in need thereof. When such compositions are in use (e.g., when a dermatological composition comprising a compound of the present disclosure) and a dermatologically acceptable excipient is placed upon the skin of the human in need thereof, the RORγt inhibitor of is in continuous contact with the skin of the patient, thereby effecting skin absorption (i.e., skin penetration) and treatment.

In topically administering the pharmaceutical compositions of the invention, the skin of the human to be treated can be optionally pre-treated (such as washing the skin with soap and water or cleansing the skin with an alcohol-based cleanser) prior to administration of the dermatological composition of the invention.

The pharmaceutical compositions of the invention may, if desired, be presented in a pack or dispenser device which may contain one or more unit dosage forms containing the active compound(s). The topical composition described herein may also be provided in a patch with the topical composition on the side of the patch that directly contacts the skin. Dermatologically acceptable adhesives may be used to affix the patch to the skin for an extended period of time.

The following Examples may be used by one skilled in the art to determine the effectiveness of the compounds of the invention in treating a human having a dermatological condition characterized by inflammation.

### EXAMPLES

### Example 1: Solubility of Various RORγt Inhibitors in Model Formulations

A series of formulations were created to test the solubilities of three RORγt inhibitors according to the present disclosure. The formulations were created to a range of systems suitable for topical application. Formulations 1-7 were created as creams with an aqueous phase, an oil phase and surfactants. Formulation 8 was created as an aqueous gel, while Formulations 8 and 19 were non-aqueous gels. Formulation 11 was a PEG-based ointment.

Each of Formulations 1-11 were used to test the solubility of three RORγt inhibitors according to the present disclosure: Compound 1, Compound 2, and Compound 3. These compounds were formulated at 80% saturated solubility in all Formulations except for the non-aqueous gels (i.e., Formulations 9 and 10), in which saturated solubility was not reached by 12%, and in order to conserve drug substance, the solvent systems were prepared at this sub-thermodynamically optimized concentration of 12%).

TABLE 1
Compositions (% w/w) of solvent systems developed for RORγT inhibitors

| | Composition of solvent systems (% w/w) | | | | | | | | | | |
|---|---|---|---|---|---|---|---|---|---|---|---|
| Excipient | 1 | 2 | 3 | 4 | 5 | 6 | 7 | 8 | 9 | 10 | 11 |
| Deionized water | 30.00 | 30.00 | 30.00 | 45.00 | 45.00 | — | 20.00 | 59.70 | — | — | — |
| PEG 300 | — | — | — | — | — | — | — | — | — | — | 30.00 |
| PEG 400 | 33.00 | 31.00 | 32.80 | 24.60 | — | — | 40.80 | — | 49.80 | 54.80 | 39.80 |
| Ethanol | — | — | — | — | — | — | — | — | 5.00 | 10.00 | — |
| Glycerol | — | — | — | — | 10.60 | — | — | — | — | 15.00 | — |
| Diethylene | 15.00 | 15.00 | 15.00 | 10.00 | — | — | 15.00 | 15.00 | 25.00 | 20.00 | — |
| Glycol | | | | | | | | | | | |
| Monoethyl Ether | | | | | | | | | | | |
| (Transcutol P) | | | | | | | | | | | |
| Propylene | — | — | — | — | 15.00 | — | — | 20.00 | 20.00 | — | — |
| Glycol | | | | | | | | | | | |
| Dimethyl | — | — | — | — | 8.00 | — | — | 5.00 | — | — | — |
| Isosorbide | | | | | | | | | | | |
| (Arlasolve) | | | | | | | | | | | |
| Sodium | — | — | 0.20 | 0.20 | 0.20 | — | 0.20 | — | — | — | — |
| metabisulfite | | | | | | | | | | | |
| Butylated | — | — | — | — | — | 0.10 | — | — | 0.20 | 0.20 | 0.20 |
| Hydroxytoluene | | | | | | | | | | | |
| (BHT) | | | | | | | | | | | |
| Benzyl Alcohol | 2.00 | 2.00 | 2.00 | — | — | — | 2.00 | — | — | — | — |
| Sodium | — | — | — | 0.20 | 0.20 | — | — | 0.20 | — | — | — |
| Benzoate | | | | | | | | | | | |
| EDTA | — | — | — | — | — | — | — | 0.10 | — | — | — |
| Isopropyl | — | — | — | — | — | 10.00 | — | — | — | — | — |
| Myristate | | | | | | | | | | | |
| Diisopropyl | — | — | — | — | — | — | 2.00 | — | — | — | 10.00 |
| Adipate | | | | | | | | | | | |
| Crodamol OHS | — | — | — | — | — | 9.90 | — | — | — | — | — |
| (Ethylexyl | | | | | | | | | | | |
| Hydroxystearate) | | | | | | | | | | | |
| Mineral Oil | — | — | — | — | — | 15.00 | — | — | — | — | — |
| Polysorbate | — | — | — | — | 1.00 | — | — | — | — | — | — |
| (Tween 20) | | | | | | | | | | | |
| Brij S20 | — | 2.00 | — | — | — | — | — | — | — | — | — |

The saturated solubility of the RORγ inhibitors was assessed in the developed solvent systems at 20° C. To conserve drug substance (i.e., Compound 1, 2 or 3), the saturated solubility experiments were capped at 10% w/w. Thus, for solvent systems in which no saturation was reached 24 h after addition of 10% w/w drug substance, results were reported as ≥10% w/w (see, Table 2 below). Compound 1, Compound 2 and Compound 3 content was assayed for each solvent system in order to generate concentration/Tin stability data. The solvent systems were placed on short-term stability and were tested at t=0 and following 2 weeks of storage at 25° C. and 50° C. The composition, solubility data, and stability for Compounds 1, 2 and 3 are detailed in Tables 2, 3, and 4 respectively.

As shown below, Compound 1 was found to be most soluble in Formulations 9 and 10, Compound 2 was most soluble in Formulations 3, 7 and 9-11, while Compound 3 was most soluble in Formulations 3 and 9-11.

TABLE 2
Compositions of solvent systems (% w/w) and saturated solubility of Compound 1

| | Composition of solvent systems (% w/w) | | | | | | | | | | |
|---|---|---|---|---|---|---|---|---|---|---|---|
| | 1 | 2 | 3 | 4 | 5 | 6 | 7 | 8 | 9 | 10 | 11 |
| Saturated | ≥1* | ≥1* | 10.26 | 1.04 | 0.87 | 0.07 | 9.7 | 0.61 | 11.4 | 13.14 | 8.92 |
| solubility of | | | | | | | | | | | |
| RORγ | | | | | | | | | | | |
| inhibitors | | | | | | | | | | | |
| (% w/w) | | | | | | | | | | | |
| Average | 86.59 | 84.78 | 48.82 | 86.91 | 70.00 | 5.38 | 150.63 | 145.46 | 97.85 | 115.91 | 153.81 |
| Concentration | | | | | | | | | | | |
| in solvent | | | | | | | | | | | |
| systems | | | | | | | | | | | |
| (μg/mL) at t = 0 | | | | | | | | | | | |
| Average | — | — | 110.05 | 100.01 | 94.79 | 111.64 | 97.70 | 98.66 | 83.55 | 83.55 | 99.94 |
| Recovery (% | | | | | | | | | | | |
| theoretical | | | | | | | | | | | |
| label claim) at | | | | | | | | | | | |
| t = 2 weeks | | | | | | | | | | | |
| 25° C. | | | | | | | | | | | |
| Average | 87.14 | 86.6 | 108.60 | 94.62 | 93.78 | 110.17 | 96.91 | 97.61 | 83.21 | 49.94 | 98.93 |
| Recovery (% | | | | | | | | | | | |
| theoretical | | | | | | | | | | | |
| label claim) at | | | | | | | | | | | |
| t = 2 weeks | | | | | | | | | | | |
| 50° C. | | | | | | | | | | | |
| Peak Purity (% | NT | NT | 99.09 | 97.89 | 98.64 | 88.39 | 98.63 | 98.79 | 98.94 | 99.06 | 98.77 |
| peak area, | | | | | | | | | | | |
| n = 1) at t = 0 | | | | | | | | | | | |
| Peak Purity (% | NT | NT | 98.63 | 93.72 | 94.97 | 89.94 | 97.47 | 94.47 | 98.62 | 98.76 | 98.51 |
| peak area, | | | | | | | | | | | |
| n = 1) t = 2 | | | | | | | | | | | |
| weeks 25° C. | | | | | | | | | | | |
| Peak Purity (% | NT | NT | 98.53 | 90.08 | 94.4 | 87.89 | 97.75 | 97.68 | 98.69 | 98.73 | 96.5 |
| peak area, | | | | | | | | | | | |
| n = 1) t = 2 | | | | | | | | | | | |
| weeks 50° C. | | | | | | | | | | | |

As shown, following 2 weeks of storage the recovery of the drug from solvent systems appeared to generally be at least 93% for the majority of systems, with the exception of Formulations 9 and 10 (ca. 83% and 49% at 50° C., respectively). It should be noted that the recoveries may reflect the fact that the samples were diluted, and thus small variations may be observed. The purity of the drug decreased slightly in the solvent systems over 2 weeks of storage, with purity of >93% being observed in all solvent systems at 50° C. (with the exception of SSCR06). The highest purity of the drug was achieved in gel solvent systems (Formulations 8, 9 and 10) which exhibited >98% purity after storage at 50° C. for 2 weeks.

TABLE 3
Compositions of solvent systems (% w/w) and saturated solubility of Compound 2

| | Composition of solvent systems (% w/w) | | | | | | | | | | |
|---|---|---|---|---|---|---|---|---|---|---|---|
| | 1 | 2 | 3 | 4 | 5 | 6 | 7 | 8 | 9 | 10 | 11 |
| Saturated | ≥1* | ≥1* | 12.54 | 0.59 | 0.75 | 0.08 | 11.55 | 0.39 | 11.89 | 12.35 | 12.18 |
| solubility of | | | | | | | | | | | |
| RORγ | | | | | | | | | | | |
| inhibitors | | | | | | | | | | | |
| (% w/w) | | | | | | | | | | | |
| Average | 109.29 | 108.26 | 30.44 | 47.86 | 62.74 | 5.83 | 101.21 | 111.59 | 97.05 | 100.34 | 110.07 |
| Concentration | | | | | | | | | | | |
| in solvent | | | | | | | | | | | |
| systems | | | | | | | | | | | |
| (μg/mL) at t = 0 | | | | | | | | | | | |
| Average | NT | NT | 97.35 | 95.04 | 89.77 | 135.06 | 78.80 | 79.60 | 79.77 | 80.31 | 53.68 |
| Recovery (% | | | | | | | | | | | |
| theoretical | | | | | | | | | | | |
| label claim) at | | | | | | | | | | | |
| t = 2 weeks | | | | | | | | | | | |
| 25° C. | | | | | | | | | | | |
| Average | 109.66 | 107.4 | 97.18 | 92.82 | 88.21 | 136.04 | 78.97 | 79.32 | 79.30 | 79.34 | 51.08 |
| Recovery (% | | | | | | | | | | | |
| theoretical | | | | | | | | | | | |
| label claim) at | | | | | | | | | | | |
| t = 2 weeks | | | | | | | | | | | |
| 50° C. | | | | | | | | | | | |
| Peak Purity (% | NT | NT | 99.2 | 97.01 | 98.56 | 96.9 | 98.32 | 98.84 | 99.18 | 99.19 | 99.33 |
| peak area, | | | | | | | | | | | |
| n = 1) at t = 0 | | | | | | | | | | | |
| Peak Purity (% | NT | NT | 98.25 | 92.78 | 96.8 | 80.66 | 95.83 | 97.36 | 98.37 | 98.42 | 98.71 |
| peak area, | | | | | | | | | | | |
| n = 1) t = 2 | | | | | | | | | | | |
| weeks 25° C. | | | | | | | | | | | |
| Peak Purity (% | NT | NT | 98.12 | 90.58 | 95.25 | 81.55 | 96.96 | 95.25 | 98.37 | 98.36 | 95.96 |
| peak area, | | | | | | | | | | | |
| n = 1) t = 2 | | | | | | | | | | | |
| weeks 50° C. | | | | | | | | | | | |

Following 2 weeks of storage the recovery of the drug from solvent systems generally appeared to be <80% for the majority of systems. The recovery was notably low in Formulation 11, at around 50% at both storage conditions. The purity of the drug appeared to decrease in the solvent systems over 2 weeks of storage, with a trend toward greater decreases being observed at the higher temperature. The highest purity of the drug was achieved in gel solvent systems (Formulations 8, 9 and 10) which exhibited >98% purity after storage at 50° C. for 2 weeks.

TABLE 4
Compositions of solvent systems (% w/w) and saturated solubility of Compound 3

| | Composition of solvent systems (% w/w) | | | | | | | | | | |
|---|---|---|---|---|---|---|---|---|---|---|---|
| | 1 | 2 | 3 | 4 | 5 | 6 | 7 | 8 | 9 | 10 | 11 |
| Saturated | ≥1* | ≥1* | 12.75 | 0.16 | 0.06 | 4.88 | 11.32 | 0.4 | 12.45 | 12.66 | 12.47 |
| solubility of | | | | | | | | | | | |
| RORγ | | | | | | | | | | | |
| inhibitors | | | | | | | | | | | |
| (% w/w) | | | | | | | | | | | |
| Average | 89.28 | 86.57 | 31.20 | 12.82 | 4.84 | 365.65 | 115.99 | 97.41 | 105.93 | 104.18 | 108.52 |
| Concentration | | | | | | | | | | | |
| in solvent | | | | | | | | | | | |
| systems | | | | | | | | | | | |
| (μg/mL) at t = 0 | | | | | | | | | | | |
| Average | NT | NT | 101.15 | 83.70 | 97.42 | 105.48 | 95.14 | 82.25 | 81.03 | 81.38 | 80.05 |
| Recovery (% | | | | | | | | | | | |
| theoretical | | | | | | | | | | | |
| label claim) at | | | | | | | | | | | |
| t = 2 weeks | | | | | | | | | | | |
| 25° C. | | | | | | | | | | | |
| Average | 87.61 | 85.27 | 100.98 | 60.01 | 82.07 | 105.16 | 94.77 | 80.55 | 77.62 | 79.22 | 76.96 |
| Recovery (% | | | | | | | | | | | |
| theoretical | | | | | | | | | | | |
| label claim) at | | | | | | | | | | | |
| t = 2 weeks | | | | | | | | | | | |
| 50° C. | | | | | | | | | | | |
| Peak Purity (% | NT | NT | 97.53 | 94.79 | 90.45 | 99.56 | 99.28 | 99.42 | 99.34 | 99.42 | 99.27 |
| peak area, | | | | | | | | | | | |
| n = 1) at t = 0 | | | | | | | | | | | |
| Peak Purity (% | NT | NT | 97.02 | 93.34 | 98.82 | 96.44 | 98.9 | 98.87 | 97.82 | 98.5 | 98.15 |
| peak area, | | | | | | | | | | | |
| n = 1) t = 2 | | | | | | | | | | | |
| weeks 25° C. | | | | | | | | | | | |
| Peak Purity (% | NT | NT | 93.57 | 81.39 | 97.16 | 97.2 | 98.6 | 98.37 | 93.57 | 96.25 | 93.17 |
| peak area, | | | | | | | | | | | |
| n = 1) t = 2 | | | | | | | | | | | |
| weeks 50° C. | | | | | | | | | | | |

Following 2 weeks of storage the recovery of the drug from solvent systems appeared to be <90% for the majority of systems, with the exception of Formulation 8, Formulation 5 (at 25° C.), and Formulations 6-7. Notably poor recoveries were observed from Formulation 4 (ca. 84% and 60% at 25 and 50° C. conditions, respectively). Like with Compounds 1 and 2, the purity of the drug decreased slightly in the solvent systems over 2 weeks of storage, with purity of >94% being observed in most solvent systems at 50° C.

### Example 2: Performance of Compounds 1, 2 and 3 in Test Formulations in sRICA

A skin Resident Immune Cell Assay (sRICA) was used to test Compounds 1, 2 and 3 in Formulations as identified in Example 1 for reduction of IL-17A protein upregulation in the Th17 sRICA model. In this model, human surgical skin waste was cultured in a transwell system, with the dermis in contact with cell culture media and the stratum corneum exposed to air. To perform the assay, each human skin sample was defatted and dermatomed to 750 μm. Next, 8 mm punch biopsies were obtained and placed in a membrane transwell. The biopsies were prepared with a barrier ring to contain the formulation and prevent leakage of the formulation. The transwells were inserted into culture wells with complete media, and a cocktail of cytokines and antibodies were added to promote Th17 skin resident immune cell polarization.

The Compounds 1, 2 and 3 were formulated at 80% saturated solubility (except for the non-aqueous gels, in which saturated solubility was not reached by 12%, and in order to conserve drug substance, the solvent systems were prepared at this sub-thermodynamically optimized concentration of 12%). 10 ul of the test solvent system was applied to topically prepared sRICA samples and allowed to penetrate the skin overnight. The Th17 activation cocktail was added the next day and media was harvest 48 hours after activation. N=6 per group. An unpaired, two-tailed students T-test was used to determined statistical significance between Th17 treated skin and treatment groups. The performance of the 3 RORγt inhibitors in Formulations representative of creams (Formulations 3-7), aqueous gels (Formulation 8), non-aqueous gels (Formulations 9-10), and ointments (Formulation 11) were evaluated.

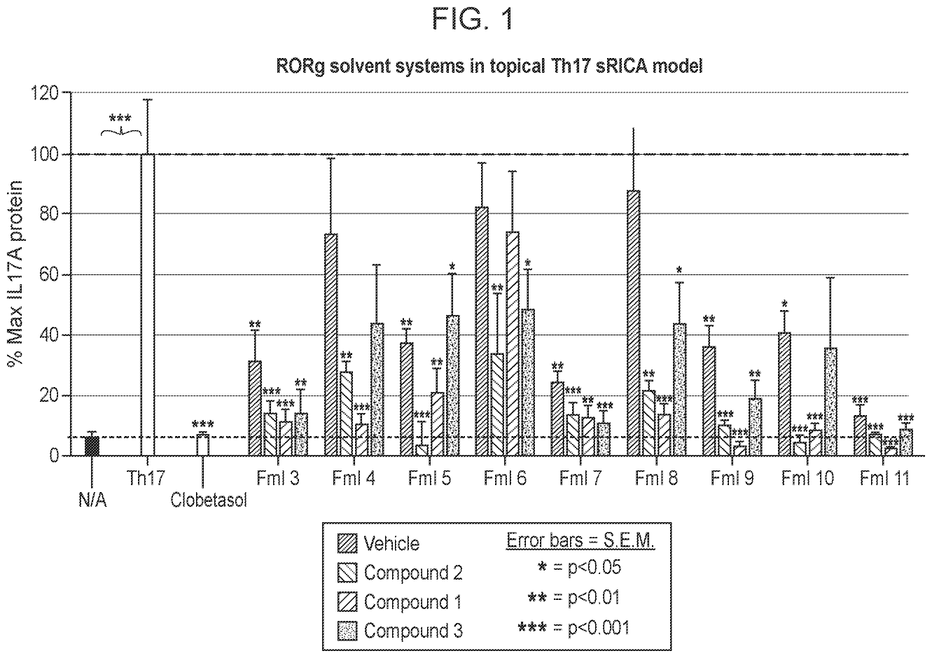

The results for this study are shown in FIG. 1. As typically occurs in the Th17 sRICA, stimulation of normal human skin with the Th17 cocktail results in robust and highly significant upregulation of IL17A protein in the culture media. Treatment of skin with topical Clobetasol (Dermovate®, blue bar) resulted in complete suppression of Th17-cocktail induced IL17A protein.

As shown, many of the lead compounds in multiple of the Formulations demonstrated significant ability to inhibit IL17A protein induction in this model, with many also performing on par with clobetasol and resulting in complete inhibition of IL17A protein (i.e., down to baseline, non-activated levels). However, several of the vehicles in this model did demonstrate significant anti-inflammatory activity. Normalization of the data to each vehicle control demonstrated that although several of the Formulations had inherent anti-inflammatory effect, the active solvent system was able to statistically separate from the vehicle control. Of note, Compound 1 in gel systems (i.e., both aqueous and non-aqueous gels, Formulations 8-10) performed extremely well.

In order to determine what component(s) in the solvent system vehicles were contributing to the large anti-inflammatory vehicle effect, vehicle effect versus the concentration of specific components found in the vehicle formulations was plotted. It was surprisingly found that the only component with trending correlation of vehicle effect was the levels of PEG400 (i.e., Pearson correlation coefficient of 0.66 and a p-value of 0.07). There was no correlation of Transcutol P, water, or propylene glycol with vehicle effects. Therefore, one focus of prototype formulation development was to reduce and/or remove PEG400, in order to obtain active formulations which better separate from placebo.

### Example 3: Evaluation of Further Test Formulations in skin Resident Immune Cell Assay (sRICA)

Further prototype formulations were evaluated in the topical Th17 sRICA as previously described. The Formulations containing active Compound 1 or Compound 2 were created according to the following in Table 5, Table 6 and Table 7.

TABLE 5
Cream Formulations Containing Compound 1

| | | Compositions (% w/w) containing active | | | | | | |
|---|---|---|---|---|---|---|---|---|
| | | Compound 1 cream formulations | | | | | | |
| | | Formulation # | | | | | | |
| | Excipient | 4 | 12 | 13 | 14 | 15 | 16 | 17 |
| Aqueous | Compound 1 | 0.66 | 2.20 | 2.20 | 2.20 | 0.89 | 0.90 | 0.54 |
| phase | Deionized water | 44.34 | 42.80 | 42.80 | 42.80 | 42.10 | 42.10 | 49.16 |
| | Super Refined | — | — | — | 13.00 | — | — | — |
| | PEG 400 | | | | | | | |
| | PEG 400 | 24.60 | 13.00 | 13.00 | — | — | — | — |
| | Sorbitol | — | — | — | — | 10.00 | 10.00 | 5.00 |
| | Diethylene | 10.00 | 20.00 | 20.00 | 20.00 | 20.00 | 20.00 | 25.00 |
| | Glycol | | | | | | | |
| | Monoethyl Ether | | | | | | | |
| | (Transcutol P) | | | | | | | |
| | Propylene glycol | — | — | — | — | 5.00 | 5.00 | — |
| | EDTA disodium | — | — | — | — | — | — | 0.10 |
| | Sodium | 0.20 | — | — | — | — | — | — |
| | metabisulfite | | | | | | | |
| | Sodium benzoate | 0.20 | — | — | — | — | — | 0.20 |
| | Benzyl alcohol | — | 2.00 | 2.00 | 2.00 | 2.00 | 2.00 | — |
| Oil phase | Isopropyl | 2.86 | 2.86 | — | — | 2.86 | — | — |
| | myristate (IPM) | | | | | | | |
| | Ethylhexyl | 2.86 | 2.86 | — | — | 2.86 | — | — |
| | Hydroxystearate | | | | | | | |
| | (Crodamol OHS) | | | | | | | |
| | Mineral oil | 4.29 | 4.29 | — | — | 4.29 | 6.00 | — |
| | Oleyl Alcohol | — | — | 4.00 | 4.00 | — | — | 4.00 |
| | (Kollicream OA) | | | | | | | |
| | Octyldodecanol | — | — | 4.00 | 4.00 | — | — | 4.00 |
| | Kollicream OD) | | | | | | | |
| | Stearic acid | 5.00 | 5.00 | — | — | 5.00 | 4.50 | — |
| | Cetyl alcohol | — | — | 8.00 | 8.00 | — | 4.50 | 8.00 |
| | (Kolliwax CA) | | | | | | | |
| Surfactant | Steareth-2 | 1.37 | 1.37 | — | — | 1.37 | — | — |
| | (Brij S2) | | | | | | | |
| | Steareth-20 | 3.63 | 3.63 | — | — | 3.63 | — | — |
| | (Brij S20) | | | | | | | |
| | Polyethylene | — | — | — | — | — | 2.75 | — |
| | Glycol | | | | | | | |
| | Hexadecyl Ether | | | | | | | |
| | (Cetomacrogol | | | | | | | |
| | 1000) | | | | | | | |
| | Cetostearyl | — | — | — | — | — | 2.25 | — |
| | Alcohol | | | | | | | |
| | (Crodacol CS90) | | | | | | | |
| | Glyceryl | — | — | 4.00 | 4.00 | — | — | 4.00 |
| | Monostearate | | | | | | | |
| | (Gelot 64) | | | | | | | |

| | | | | | | | |
|---|---|---|---|---|---|---|---|
| Total | 100.00 | 100.00 | 100.00 | 100.00 | 100.00 | 100.00 | 100.00 |

TABLE 6
Aqueous and Non-Aqueous Gel Formulations Containing Compound 1

| | | Composition (% w/w) of active Compound 1 gel formulations | | | | | |
|---|---|---|---|---|---|---|---|
| | | Formulation # | | | | | |
| | Excipient | 8 | 18 | 19 | 10 (1%) | 10 (5%) | 20 |
| Aqueous | Compound 1 | 0.49 | 3.28 | 0.82 | 1.00 | 5.00 | 5.00 |
| Phase | Deionised water | 57.21 | 44.88 | 52.78 | — | — | — |
| | Ethanol | — | — | — | 9.70 | 9.30 | 9.40 |
| | PEG 400 | — | — | — | 53.15 | 50.96 | — |
| | Glycerol | — | — | 5.00 | 14.55 | 13.95 | 18.80 |
| | Sorbitol | — | 9.53 | — | — | — | — |
| | Diethylene Glycol | 15.00 | 19.05 | 20.00 | 19.40 | 18.60 | 46.81 |
| | Monoethyl Ether | | | | | | |
| | (Transcutol P) | | | | | | |
| | Propylene glycol | 20.00 | 19.05 | 20.00 | — | — | 18.80 |
| | Dimethyl | 5.00 | — | — | — | — | — |
| | isosorbide (DMI) | | | | | | |
| | EDTA disodium | 0.10 | — | 0.10 | — | — | — |
| | Sodium | — | 0.20 | — | — | — | — |
| | metabisulfite | | | | | | |
| | Butylated | — | — | — | 0.20 | 0.20 | 0.20 |
| | hydroxytoluene | | | | | | |
| | (BHT) | | | | | | |
| | Ascorbic acid | — | — | 0.10 | — | — | — |
| | Sodium benzoate | 0.20 | — | 0.20 | — | — | — |
| | Benzyl alcohol | — | 2.00 | — | — | — | — |
| Polymer | Hydroxypropyl | 2.00 | 2.00 | — | — | — | — |
| | methyl cellulose | | | | | | |
| | (Benecel E4M) | | | | | | |
| | Carbopol 980 | — | — | 1.00 | — | — | — |
| | Hydroxypropyl | — | — | — | 2.00 | 2.00 | — |
| | cellulose (Klucel | | | | | | |
| | MF) | | | | | | |
| | Hydroxypropyl | — | — | — | — | — | 1.00 |
| | cellulose (Klucel | | | | | | |
| | HF) | | | | | | |

| | | | | | | |
|---|---|---|---|---|---|---|
| Total | 100.00 | 100.00 | 100.00 | 100.00 | 100.00 | 100.00 |

TABLE 7
Cream and Non-Aqueous Gel Formulations
Containing Compound 2

| | | Composition (% w/w) of active | |
|---|---|---|---|
| | | Compound 2 cream and gel formulations | |
| | | Formulation # | |
| | Excipient | 21 | 10 |
| Aqueous | Compound 2 | 0.49 | 5.00 |
| phase | Deionized water | 44.51 | — |
| | Ethanol | — | 9.30 |
| | PEG 400 | — | 50.96 |
| | Glycerol | 10.60 | 13.95 |
| | Diethylene Glycol | — | 18.60 |
| | Monoethyl Ether | | |
| | (Transcutol P) | | |
| | Propylene Glycol | 15.00 | — |
| | Dimethyl | 8.00 | — |
| | isosorbide (DMI) | | |
| | Sodium | 0.20 | — |
| | Metabisulfite | | |
| | Butylated | — | 0.20 |
| | hydroxytoluene | | |
| | (BHT) | | |
| | Sodium benzoate | 0.20 | — |
| Oil phase | Isopropyl | 4.57 | — |
| | myristate (IPM) | | |
| | Ethylhexyl | 4.57 | — |
| | hydroxystearate | | |
| | (Crodamol OHS) | | |
| | Mineral oil | 6.86 | — |
| Surfactant | Sepineo P600 | 4.00 | — |
| | Polysorbate 20 | 1.00 | — |
| | (Tween 20) | | |
| Polymer | Hydroxypropyl | — | 2.00 |
| | cellulose (Klucel | | |
| | MF) | | |

| | | |
|---|---|---|
| Total | 100.00 | 100.00 |

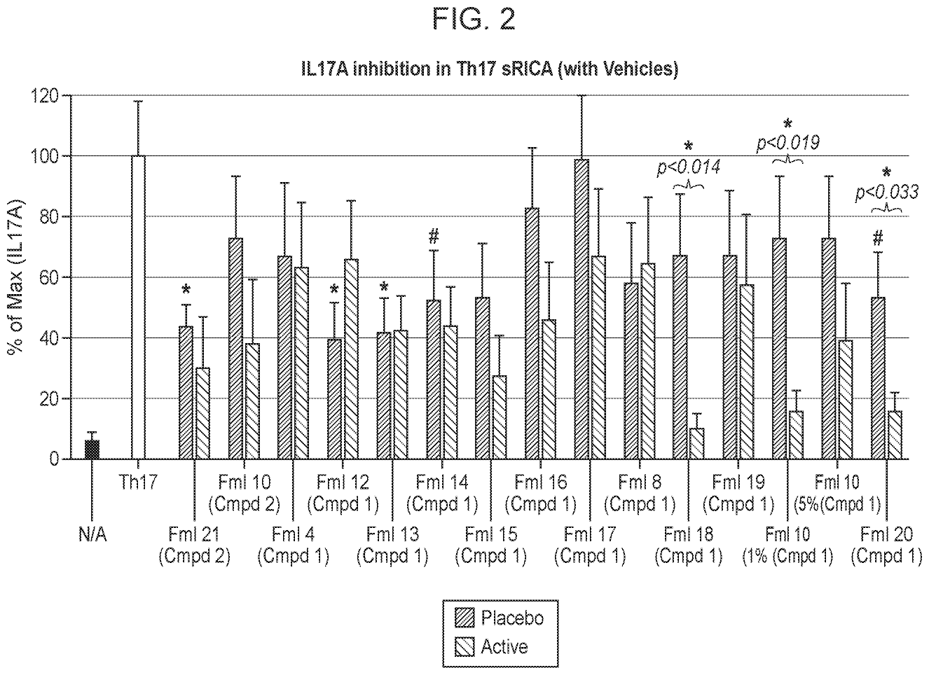

All formulations of containing Compound 1 or Compound 2 were created at 80% saturation, except for all non-aqueous gels (NAG) in which saturation has not yet been determined (>12%) and were arbitrarily formulated at 1% or 5% (see Formulation 10 (1%) and Formulation 10 (5%) in Tables 6 and 7). 10 μl of each formulation (active or vehicle) was applied to topically prepared biopsies. Topical products were given overnight to penetrate the skin prior to adding the inflammatory Th17 cocktail. Conditioned cell media was harvested 48 hours after inflammation and IL17A protein level was quantified. Combined results of 3 donors, performed in triplicate (N=9) are summarized in FIG. 2. An unpaired, two-tailed students T-test was used to determined statistical significance between vehicle versus active treated skin. Red font indicated statistical difference of vehicle vs Th17 activation alone. #p<0.1, *p<0.05. Error bars=S.E.M.

Although the reduction and/or removal of PEG400 from formulations reduced the anti-inflammatory effect of the vehicles, unfortunately there was also a corresponding loss of efficacy, particularly noted for the cream formulations. However, Compound 1 in several gel formulations (Formulation 8, Formulation 10, Formulation 20) performed well, demonstrating the ability to completely suppress IL17A protein expression and also having significant statistical separation from placebo/vehicle only control. In particular, Formulation 4 showed notably good results, performing within 5% of clobetasol.

Further studies were carried to observe the stability characteristics of the Formulations in Tables 5-7. At the time of creation, active cream formulations containing Compound 1 (Formulations in Table 5) appeared white, opaque and ranged from low to high visual viscosity, with smooth application. However, some Formulations were found to be stringy (Formulations 4 and 13). Following storage at 25° C., some formulations exhibited slight color change of white to off-white (Formulations 4, 13 and 16) and Formulation 4 became non-pourable and had a high viscosity. Notably, Formulation 12 was observed to phase separate. At the higher temperature, similar color changes were observed (white to off-white) in Formulations 12, 14 and 16. The cream containing Compound 2 (Formulation 21) showed a change in color after 2 weeks at both 25 and 40° C. (white to off-white at 25° C. and white to faint grey at 40° C.).

On the other hand, the gel formulations containing Compound 1 did not exhibit much change in appearance after 2 weeks of storage, though some changes in visual viscosity were observed (Formulation 10 1% active appeared to be of medium viscosity at 40° C. as compared to high viscosity at t=0) and a change in color from faint yellow to dark yellow was observed in Formulation 19. Formulation 10 containing Compound 1 at 5% did not appear to change from t=0.

### Example 4: Evaluation of Aqueous Gel Formulations

Of the prototypes evaluated, 3% Compound 1 in Aqueous Gel Formulation 8 made without benzyl alcohol consistently showed superior solubility, stability, efficacy (via the sRICA model), and patient acceptance. However, given the high level of water in the formulation, a preservative is anticipated to be necessary to prevent microbial growth. However, when 3% in Aqueous Gel Formulation 8 was prepared with 2% benzyl alcohol, precipitation was observed. Additionally, AG04 had an apparent pH of 3-4, which, being less than skin pH has the potential to be irritating when applied to broken skin. Studies were therefore performed to evaluate the effect of various levels of benzyl alcohol, and of various additional or substitute preservatives, gelling agents, and humectants for their effect on compound solubility and formulation turbidity. Formulations having high solubility and improved appearance were further evaluated for chemical and physical stability, including appearance and pH. A summary of the compositions placed on stability are presented in Table 8.

TABLE 8
Aqueous Gel Formulations

| | | Composition of formulations (% w/w) | | | | | | |
|---|---|---|---|---|---|---|---|---|
| | | Formulation | | | | | | |
| | Excipient | 8 | 22 | 23 | 24 | 25 | 26 | 27 |
| Aqueous | Compound 1 | 3.35 | 2.900 | 3.028 | 3.115 | 3.028 | 2.935 | 3.028 |
| phase | Deionised water | 45.80 | 45.89 | 45.34 | 44.24 | 45.83 | 45.74 | — |
| | Citrate buffer, | — | — | — | — | — | — | 45.34 |
| | pH 5 | | | | | | | |
| | Sorbitol | 9.73 | 9.50 | — | — | — | 9.71 | — |
| | Glycerol | — | — | 9.49 | 9.87 | 9.59 | — | 9.49 |
| | Transcutol P | 19.44 | 19.00 | 18.97 | 19.74 | 19.18 | 19.43 | 18.97 |
| | Propylene glycol | 19.44 | 19.00 | 18.97 | 19.73 | 19.18 | 19.44 | 18.97 |
| | Sodium | 0.20 | 0.20 | 0.20 | 0.21 | 0.20 | 0.20 | 0.20 |
| | metabisulfite | | | | | | | |
| | Benzyl alcohol | — | 1.50 | 2.00 | 2.06 | 2.00 | 1.52 | 2.00 |
| pH | 0.2M NaOH in water | — | — | — | Adjust | — | Adjust | — |
| adjustment | | | | | to pH | | to pH | |
| | | | | | 5.5-6.0 | | 5.5-6.0 | |
| | Deionised | — | — | — | Q.S. to | — | Q.S. to | — |
| | water, 2^{nd} addition | | | | 100% | | 100% | |
| Polymer | Hydroxypropyl | 2.04 | 2.00 | 2.00 | — | — | — | 2.00 |
| | Methyl Cellulose | | | | | | | |
| | (Benecel E4M) | | | | | | | |
| | Carbopol 980 | — | — | — | 1.03 | | 1.02 | — |
| | Hydroxyethyl | — | — | — | — | 1.00 | — | — |
| | Cellulose (Natrosol | | | | | | | |
| | 250) | | | | | | | |

| | | | | | | | |
|---|---|---|---|---|---|---|---|
| Total | 100.00 | 100.00 | 100.00 | 100.00 | 100.00 | 100.00 | 100.00 |

All formulations were found to be stable, even following 4 weeks of storage at 40° C., exhibiting less than 5% loss of recovery after that period of time. Drug purity was also good, exhibiting only a slight decrease over time, but was within ±0.5% of the value obtained at t=0 after 4 weeks of storage at 40° C. Additionally, all Formulations in Table 8 appeared to maintain color and consistent viscosity over the entire testing period. Additionally, no particulates were observed microscopically in any of the formulations, suggesting that physical stability of the drug and polymer in the formulation was achieved.

At t=0, the apparent pH of active formulations containing Compound 1 ranged from 4.39-5.62 and following 3 days of storage at 25° C. the apparent pH was broadly consistent with t=0 (±0.5 pH units). After 4 weeks of storage at 25 and 40° C., a slight downward trend was observed in the formulations which were not pH adjusted or buffered, suggesting that the citrate buffer employed in Formulation 27 or the pH adjustment performed for Formulations 24 and 26 were sufficient to stabilize pH over time. Without being bound by theory, it is thought that sodium metabisulfite, a component which is in all of the formulation may be contributing to the decrease in apparent pH over time.

Formulation 22 (1.5% benzyl alcohol) was designed with lower levels of benzyl alcohol than originally included in Formulation 8 (2%). The resultant formulations were generally slightly translucent, however Formulation 22 showed turbidity over the test period. Without being bound by theory, it is possible that the turbidity is indicative of gelling agent that has not fully solvated on the small scale that it was prepared at (20 g) but as the effect was more pronounced with the higher levels of benzyl alcohol, it suggests a cause due to the combination of benzyl alcohol and sorbitol. An alternative humectant, glycerol, with 2% w/w benzyl alcohol was also prepared and this did not exhibit any turbidity, suggesting that sorbitol may be responsible for turbidity.

Formulations 24-26 were prepared with buffers and different gelling agents, with 2% benzyl alcohol and glycerol (Formulations 24 and 25) and 1.5% benzyl alcohol and sorbitol (Formulation 26). Carbopol 980, which requires neutralization (i.e. pH adjustment) to hydrate was employed in Formulation 24 and Formulation 26, which were both clear and colorless. Formulation 25, which included HEC, appeared to be clear and no precipitation was observed.

The 3% Compound 1 in Aqueous Gel Formulation 23 demonstrated good chemical/physical stability. Moreover, Formulation 23 formulation differed only slightly from the Formulation 8 that had previously demonstrated acceptable chemical/physical stability, sRICA data, and patient acceptance; Formulation 23 contained 2% benzyl alcohol and glycerol instead of sorbitol. Therefore, Formulation 23 was selected to be scaled up for non-GLP tox batches.

Formulations 8, 22 and 23 were also evaluated using sRICA following the same methods described above. Results from the initial prototype formulation screening of Formulation 8 discussed in Example 3, were repeated and tested with a new batch for formulation. Formulation 8 consistently performed well in the Th17 sRICA versus vehicle. Formulation 10, which contains 1.5% benzyl alcohol, was found to perform just as well as Formulation 8, demonstrating that benzyl alcohol does not affect the efficacy of aqueous gels containing Compound 1. For Composition 23, the humectant sorbitol was replaced with glycerol. Similarly, Composition 23 demonstrated efficacy on par with Formulation 8 and Formulation 22, and statistically separated from placebo. Formulation 23 performed better than Formulations 24-26, as well.

### Example 5: Toxicology Studies of Aqueous Gel Formulations

Formulation 23 (containing 3.028% w/w Compound 1) as well as Formulation 23 placebo were assessed at t=0 and following 1 and 5 months of storage at 25 and 40° C. The samples were assessed for recovery and purity of the drug, apparent pH, visual appearance, microscopic appearance, apparent viscosity (assessed via Brookfield viscometer), MQT and PET.

Results are summarized below

TABLE 9
Summary of formulation stability assessment of Formulation
23 Containing Compound 1 after 1 Month

| | Formulation stability assessment of | | |
|---|---|---|---|
| | Formulation 23 (3.028% w/w Compound 1) | | |
| | t = 0 | t = 1 month | |
| Test parameter | (release testing) | 25° C. | 40° C. |
| Mean recovery of | 96.07 | 100.34 | 99.83 |
| Compound 1 as a | | | |
| percentage of the | | | |
| label claim, n = 3 | | | |
| Mean purity | 97.37 | 97.52 | 97.40 |
| (expressed as % | | | |
| Compound 1 area) | | | |
| of drug, n = 3 | | | |
| Mean apparent pH, | 4.46 | 3.87 | 3.45 |
| n = 3 | | | |
| Visual appearance | Clear, colourless | No obvious | No obvious |
| | gel of smooth | change | change |
| | application with | from t = 0 | from t = 0 |
| | a medium visual | | |
| | viscosity | | |
| Microscopic | No excipient | No obvious | No obvious |
| appearance (light | particulates or | change | change |
| microscopy, 400× | API crystals | from t = 0 | from t = 0 |
| magnification), | observed | | |
| n = 3 | | | |
| Apparent viscosity | 79,210 mPa · s | 93,898 mPa · s | 86,086 mPa · s |
| (Brookfield | Torque 50.7% | Torque 60.1% | Torque 55.1% |
| viscometer, Spindle | | | |
| E, 3 rpm) | | | |

TABLE 10
Summary of formulation stability assessment of Formulation
23 Containing Compound 1 after 3 Months

| | Formulation stability assessment of | | |
|---|---|---|---|
| | Formulation 23 (3.028% w/w Compound 1) | | |
| | t = 0 | t = 3 months | |
| Test parameter | (release testing) | 25° C. | 40° C. |
| Mean recovery of | 96.07 | 96.81 | 98.24 |
| Compound 1 as a | | | |
| percentage of the | | | |
| label claim, n = 3 | | | |
| Mean purity | 97.37 | 97.06 | 97.13 |
| (expressed as % | | | |
| Compound 1 area) | | | |
| of drug, n = 3 | | | |
| Mean apparent pH, | 4.46 | 3.66 | 3.95 |
| n = 3 | | | |
| Visual appearance | Clear, colourless | No obvious | No obvious |
| | gel of smooth | change | change |
| | application with | from t = 0 | from t = 0 |
| | a medium visual | | |
| | viscosity | | |
| Microscopic | No excipient | No obvious | No obvious |
| appearance (light | particulates or | change | change |
| microscopy, 400× | API crystals | from t = 0 | from t = 0 |
| magnification), | observed | | |
| n = 3 | | | |
| Apparent viscosity | 79,210 mPa · s | 84,060 mPa · s | 80,150 mPa · s |
| (Brookfield | Torque 50.7% | Torque 53.8% | Torque 51.3% |
| viscometer, Spindle | | | |
| E, 3 rpm) | | | |

TABLE 11
Summary of formulation stability assessment of Formulation
23 Containing Compound 1 after 5 Months

| | Formulation stability assessment of | | |
|---|---|---|---|
| | Formulation 23 (3.028% w/w Compound 1) | | |
| | t = 0 | t = 5 months | |
| Test parameter | (release testing) | 25° C. | 40° C. |
| Mean recovery of | 96.07 | 99.86 | 99.83 |
| Compound 1 as a | | | |
| percentage of the | | | |
| label claim, n = 3 | | | |
| Mean purity | 97.37 | 97.05 | 97.11 |
| (expressed as % | | | |
| Compound 1 area) | | | |
| of drug, n = 3 | | | |
| Mean apparent pH, | 4.46 | 3.91 | 3.66 |
| n = 3 | | | |
| Visual appearance | Clear, colourless | No obvious | No obvious |
| | gel of smooth | change | change |
| | application with | from t = 0 | from t = 0 |
| | a medium visual | | |
| | viscosity | | |
| Microscopic | No excipient | No obvious | No obvious |
| appearance (light | particulates or | change | change |
| microscopy, 400× | API crystals | from t = 0 | from t = 0 |
| magnification), | observed | | |
| n = 3 | | | |
| Apparent viscosity | 79,210 mPa · s | 93,960 mPa · s | 82,210 mPa · s |
| (Brookfield | Torque 50.7% | Torque 60.1% | Torque 57.1% |
| viscometer, Spindle | | | |
| E, 3 rpm) | | | |

TABLE 12
Summary of formulation stability assessment
of Formulation 23 Placebo after 1 Month

| | Formulation stability assessment of | | |
|---|---|---|---|
| | Formulation 23 vehicle | | |
| | t = 0 | t = 1 month | |
| Test parameter | (release testing) | 25° C. | 40° C. |
| Mean apparent pH | 4.25 | 3.89 | 3.53 |
| of Formulation 23, | | | |
| n = 3 | | | |
| Visual appearance | Clear, colourless | No obvious | No obvious |
| | gel of smooth | change | change |
| | application with | from t = 0 | from t = 0 |
| | a medium visual | | |
| | viscosity | | |
| Microscopic | No excipient | No obvious | No obvious |
| appearance (light | particulates or | change | change |
| microscopy, 400× | API crystals | from t = 0 | from t = 0 |
| magnification), | observed | | |
| n = 3 | | | |
| Apparent viscosity | 72,650 mPa · s | 71,867 mPa · s | 73,744 mPa · s |
| (Brookfield | Torque 46.5 | Torque 46.0% | Torque 47.2% |
| viscometer, Spindle | | | |
| E, 3 rpm) | | | |

TABLE 13
Summary of formulation stability assessment
of Formulation 23 Placebo after 3 Months

| | Formulation stability assessment of | | |
|---|---|---|---|
| | Formulation 23 vehicle | | |
| | t = 0 | t = 3 months | |
| Test parameter | (release testing) | 25° C. | 40° C. |
| Mean apparent pH | 4.25 | 3.96 | 3.99 |
| of Formulation 23, | | | |
| n = 3 | | | |
| Visual appearance | Clear, colourless | No obvious | No obvious |
| | gel of smooth | change | change |
| | application with | from t = 0 | from t = 0 |
| | a medium visual | | |
| | viscosity | | |
| Microscopic | No excipient | No obvious | No obvious |
| appearance (light | particulates or | change | change |
| microscopy, 400× | API crystals | from t = 0 | from t = 0 |
| magnification), | observed | | |
| n = 3 | | | |
| Apparent viscosity | 72,650 mPa · s | 83,280 mPa · s | 86,250 mPa · s |
| (Brookfield | Torque 46.5 | Torque 53.3% | Torque 55.2% |
| viscometer, Spindle | | | |
| E, 3 rpm) | | | |

TABLE 14
Summary of formulation stability assessment
of Formulation 23 Placebo after 5 Months

| | Formulation stability assessment of | | |
|---|---|---|---|
| | Formulation 23 vehicle | | |
| | t = 0 | t = 5 months | |
| Test parameter | (release testing) | 25° C. | 40° C. |
| Mean apparent pH | 4.25 | 3.96 | 3.59 |
| of Formulation 23, | | | |
| n = 3 | | | |
| Visual appearance | Clear, colourless | Slightly higher | Slightly higher |
| | gel of smooth | visual | visual |
| | application with | viscosity | viscosity |
| | a medium visual | | |
| | viscosity | | |
| Microscopic | No excipient | No obvious | No obvious |
| appearance (light | particulates or | change | change |
| microscopy, 400× | API crystals | from t = 0 | from t = 0 |
| magnification), | observed | | |
| n = 3 | | | |
| Apparent viscosity | 72,650 mPa · s | 93,430 mPa · s | 75,310 mPa · s |
| (Brookfield | Torque 46.5 | Torque 59.8% | Torque 48.2% |
| viscometer, Spindle | | | |
| E, 3 rpm) | | | |

Following 1 month of storage at both 25° C. and 40° C. conditions, results for Formulation 23 active and placebo were found to be consistent with t=0, with the exception of apparent pH in which a slight decrease was observed following 1 month, thought to be due to sodium metabisulfite and is concurrent with observations made above.

At 3 months a small difference in pH observed at 1 month remained and there was no major change compared to t=0 for macroscopic and microscopic observations or Compound 1 content and purity. However, there was a slight decrease in apparent viscosity in Formulation 23 containing Compound 1 (ca. 10,000 mPa·s at 25° C. and ca. 6,000 mPa·s at 40° C.). In contrast, Formulation 23 placebo increased in viscosity (ca. 12,000 mPa·s at 25° C. and ca. 13,000 mPa·s at 40° C.).

Following 5 months of storage there was no notable difference in Compound 1 recovery or purity, microscopic appearance and apparent pH compared to t=0 or the previous timepoint. However, both placebo and active formulations were of visually higher viscosity. Brookfield viscosity testing of both active and placebo formulation stored at 25° C. revealed a slight increase in viscosity (e.g. after 5 months, viscosity of AG12 active was 93,430 mPa·s compared to 72,650 mPa·s at t=0). Conversely, formulations stored at 40° C. were found to be of similar viscosity to t=0 despite being of higher visual viscosity than t=0 (i.e. non-pourable). Microbial quality tests were performed on the formulations stored at 25° C. and total aerobic microbial count and total yeast microbial count were <1.0E1 cfu/g for both active and placebo. Additionally, P. aeruginosa and S. aureus were not isolated in 1 g of the formulations.

### Example 6: Analysis of Varying the Level of Humectant

Solvent systems based on Formulation 23 were designed, manufactured and assessed for solubility of Compound 1. The composition of the solvent systems, and the results of the saturated solubility are detailed in Table 15 below. Propylene glycol, in some applications, may be capable of causing irritation to the skin. Thus, the solvent systems in Table 15 were designed with varying amounts of propylene glycol in order to assess the impact of propylene glycol on solubility in solvent systems containing water (Formulations 29, 30, and 33-35) or pH 5.0 citrate buffer (i.e., 41% citric acid solution; Formulations 31 and 32). As shown, Formulations 29-36 were formulated with propylene glycol content in range of 5-15% (i.e., lower than in Formulation 23, which contains ca. 20% w/w propylene glycol).

TABLE 15
Analysis of Solubility Relative to Concentration of Polypropylene Glycol

| | Composition of solvent systems (% w/w) | | | | | | | | |
|---|---|---|---|---|---|---|---|---|---|
| Formulation | 28 | 29 | 30 | 31 | 32 | 33 | 34 | 35 | 36 |
| Deionised | 47.80 | 53.00 | 58.00 | — | — | 53.00 | 53.00 | 48.00 | 48.00 |
| water | | | | | | | | | |
| pH 5 buffer | — | — | — | 53.00 | 58.00 | — | — | — | — |
| Glycerol | 10.00 | 10.00 | 10.00 | 10.00 | 10.00 | 10.00 | 10.00 | 10.00 | 10.00 |
| Diethylene | 20.00 | 30.00 | 20.00 | 30.00 | 20.00 | 25.00 | 20.00 | 25.00 | 40.00 |
| Glycol | | | | | | | | | |
| Monoethyl | | | | | | | | | |
| Ether | | | | | | | | | |
| (Transcutol P) | | | | | | | | | |
| Propylene | 20.00 | 5.00 | 10.00 | 5.00 | 10.00 | 10.00 | 15.00 | 15.00 | — |
| glycol | | | | | | | | | |
| Benzyl | 2.00 | 2.00 | 2.00 | 2.00 | 2.00 | 2.00 | 2.00 | 2.00 | 2.00 |
| alcohol | | | | | | | | | |
| Sodium | 0.20 | — | — | — | — | — | — | — | — |
| metabisulfite | | | | | | | | | |
| Total | 100.00 | 100.00 | 100.00 | 100.00 | 100.00 | 100.00 | 100.00 | 100.00 | 100.00 |
| Saturated | 3.78 | 4.61 | 0.52 | 4.26 | 0.55 | 2.06 | 1.22 | 4.98 | 13.01% |
| solubility of | | | | | | | | | |
| Compound 1 | | | | | | | | | |
| (% w/w) | | | | | | | | | |

As shown above, the non-solvent effect of water/buffer was evident in Formulation 23 and Formulation 32, where a slight decrease in propylene glycol and Transcutol P content decreased the solubility of Compound 1 to ca. 0.5% w/w. Similar or superior solubility of Compound 1 to Formulation 28 was achieved in Formulation 29 and Formulation 30 (5% propylene glycol, 30% Transcutol P) and Formulation 35 (15% propylene glycol, 25% Transcutol P) and Formulation 36 (0% propylene glycol, 40% Transcutol P). The inclusion of buffer in place of deionised water did not appear to have a large impact on Compound 1 solubility when Formulations 29 and 30 were compared to Formulations 31 and 32, respectively.

The results suggest that lower levels of propylene glycol may be introduced into formulation while maintaining comparable drug loading of Compound 1 to Formulation 23, if the levels of Transcutol P are increased.

### Example 7: Evaluation of Alternative Antioxidants

In order to further stabilize the pH in the formulation, placebo formulations with alternative antioxidant to sodium metabisulfite were prepared, since sodium metabisulfite appeared to create a decrease in the formulations' pH over time. Due to the high aqueous content of the formulations, water soluble antioxidants were included in the composition detailed in Table 57 where the macroscopic/microscopic appearance and apparent pH, are presented.

TABLE 16
Composition of placebo formulation containing
alternative antioxidants and resulting pH

| | | Composition of placebo | | | |
|---|---|---|---|---|---|
| | | formulations (% w/w) | | | |
| | | Formulation # | | | |
| | Excipient | 23 | 37 | 38 | 39 |
| Aqueous | Deionised | 46.82 | 55.90 | 55.99 | — |
| Phase | water | | | | |
| | pH 5.0 buffer | — | — | — | 50.90 |
| | Glycerol | 9.80 | 10.00 | 10.00 | 10.00 |
| | Diethylene | 19.59 | 20.00 | 20.00 | 30.00 |
| | Glycol | | | | |
| | Monoethyl | | | | |
| | Ether | | | | |
| | (Transcutol P) | | | | |
| | Propylene | 19.59 | 10.00 | 10.00 | 5.00 |
| | glycol | | | | |
| | Sodium | 0.20 | — | — | — |
| | metabisulfite | | | | |
| | Ascorbic acid | — | 0.10 | — | 0.10 |
| | Propyl gallate | — | — | 0.01 | — |
| | Benzyl alcohol | 2.00 | 2.00 | 2.00 | 2.00 |
| Polymer | Hydroxypropyl | 2.00 | 2.00 | 2.00 | 2.00 |
| | Methyl | | | | |
| | Cellulose | | | | |
| | (Benecel E4M) | | | | |
| | Total | 100.00 | 100.00 | 100.00 | 100.00 |
| Apparent pH | 3.88** | 3.88 | 6.79 | 5.33 | 3.88** |

Each of Formulations 37-39 were created with alternative antioxidants (i.e., ascorbic acid, propyl gallate), and Formulation 30 additionally included a pH 5.0 buffer. Each of the formulations were clear and colorless, and exhibited low viscosity and smooth application after they were made.

The inclusion of ascorbic acid in Formulation 37 was observed to lower the pH of the formulation (3.88), while the pH of Formulation 38 containing propyl gallate was 6.79, but both ascorbic acid and propyl gallate were both observed to be physically stable in Formulations 37 and 38. To increase the formulation pH, a buffered solvent system was employed in Formulation 39. The pH of the formulation was successfully buffered to 5.33. It should be noted that maximal stability of ascorbic acid in solution is achieved at around pH 5.4.

These results suggest that sodium metabisulfite can successfully be substituted for another antioxidant (e.g. propyl gallate), but that it may be necessary to buffer the formulation if an acidic antioxidant (e.g., ascorbic acid) is employed. Following these results, further compositions were created, as outlined in Table 17.

TABLE 17
Composition of active formulations containing
decreased levels of propylene glycol and alternative
antioxidants to sodium metabisulfite

| | Composition of formulations (% w/w) | | | |
|---|---|---|---|---|
| | Formulation # | | | |
| Excipient | 23 | 40 | 41 | 42 |
| Compound 1 | 3.028 | 6.24 | 3.68 | 3.03 |
| Deionised water | 45.34 | 48.52 | 49.92 | 45.51 |
| Glycerol | 9.49 | 9.15 | 9.42 | 9.48 |
| Diethylene Glycol | 18.97 | 32.04 | 28.26 | 18.96 |
| Monoethyl Ether | | | | |
| (Transcutol P) | | | | |
| Propylene glycol | 18.97 | — | 4.71 | 18.96 |
| Sodium metabisulfite | 0.20 | — | — | — |
| Propyl gallate | — | 0.05 | — | 0.05 |
| Alpha-tocopherol | — | — | 0.002 | — |
| (vitamin E) | | | | |
| Benzyl alcohol | 2.00 | 2.00 | 2.00 | 2.00 |
| Hydroxypropyl | 2.00 | 2.00 | 2.00 | 2.00 |
| Methyl Cellulose | | | | |
| (Benecel E4M) | | | | |
| Total | 100.00 | 100.00 | 100.00 | 100.00 |

Formulation 40 was created without propylene glycol and sodium bisulfite was replaced with propyl gallate. Formulation 41 was created with reduced propylene glycol, and sodium bisulfite was replaced with alpha-tocopherol acetate. Formulation 42 had a high propylene glycol content, but sodium bisulfite was replaced with propyl gallate.

TABLE 18
Recovery of Compound 1 after 4 Weeks

| | Percentage recovery (%) of Compound 1 | | | | |
|---|---|---|---|---|---|
| | (as a percentage of the theoretical concentration) | | | | |
| | | t = 2 weeks | | t = 4 weeks | |
| Formulation | t = 0 | 25° C. | 40° C. | 25° C. | 40° C. |
| 23 | 99.40 | 98.59 | 99.45 | 96.52 | 96.02 |
| 40 | 98.64 | 96.48 | 96.50 | 97.28 | 97.98 |
| 41 | 100.11 | 96.03 | 96.38 | 96.25 | 96.83 |
| 42 | 101.98 | 97.03 | 96.62 | 96.37 | 97.54 |

TABLE 19
Purity of Compound 1 after 4 Weeks

| | Purity (%) of Compound 1 | | | | |
|---|---|---|---|---|---|
| | (as a percentage of the theoretical concentration) | | | | |
| | | t = 2 weeks | | t = 4 weeks | |
| Formulation | t = 0 | 25° C. | 40° C. | 25° C. | 40° C. |
| 23 | 97.47 | 97.44 | 97.33 | 97.17 | 97.10 |
| 40 | 97.73 | 97.75 | 97.74 | 97.70 | 97.71 |
| 41 | 97.80 | 97.77 | 97.74 | 97.74 | 97.74 |
| 42 | 97.80 | 97.80 | 97.77 | 97.77 | 97.76 |

As shown in Tables 18 and 19, at t=0 all the formulations had drug recoveries of ca. 98.64-101.98%. The drug recovery remained 100%±5% after 4 weeks at both 25 and 40° C., with no obvious trends suggesting drug chemical instability. Purity of the drug following storage for 4 weeks at both storage conditions was consistent with t=0 (i.e. between 97.71-97.77%).

The developed formulations exhibited slightly improved drug recovery and purity to Formulation 23, and the data suggests that the substitution of sodium metabisulfite for an alternative antioxidant (i.e. propyl gallate or vitamin E) had no adverse impact on drug chemical stability. Furthermore, the inclusion or exclusion of propylene glycol from the formulation does not appear to have a notable impact on chemical stability of the drug following storage for up to 4 weeks.

TABLE 19
Apparent pH of Compound 1-containing formulations
at t = 0 and following 4 weeks of storage

| | Percentage recovery (%) of Compound 1 | | | | |
|---|---|---|---|---|---|
| | (as a percentage of the theoretical concentration) | | | | |
| | | t = 2 weeks | | t = 4 weeks | |
| Formulation | t = 0 | 25° C. | 40° C. | 25° C. | 40° C. |
| 23 (Placebo) | 3.88 | 3.75 | 2.96 | 3.69 | 2.83 |
| 23 (Active) | 4.33 | 3.84 | 3.24 | 3.66 | 3.13 |
| 40 (Placebo) | 6.16 | 6.85 | 6.20 | 5.60 | 6.10 |
| 40 (Active) | 6.29 | 6.16 | 5.41 | 5.70 | 5.18 |
| 41 (Placebo) | 6.27 | 6.77 | 6.16 | 6.51 | 6.10 |
| 41 (Active) | 6.65 | 6.21 | 5.72 | 5.84 | 5.06 |
| 42 (Placebo) | 6.57 | 6.68 | 6.27 | 6.42 | 6.15 |
| 42 (Active) | 6.43 | 6.28 | 5.71 | 6.00 | 5.69 |

As shown in Table 19, at t=0 the apparent pH was higher in Formulations 40-41 than Formulation 23, which is to be anticipated due to the removal of sodium metabisulfite. Following 2 weeks of storage, formulations were within 1 pH unit from that reported at t=0, however it should be noted that active formulation stored at 40° C. exhibited a downward trend, but remained notably higher in pH than AG12. This continued into the 4 week time point and this decrease in apparent pH from AG28 and AG35 stored at 40° C. was greater than 1 (1.11 and 1.59, respectively).

As no obvious drop in drug purity was observed in Section 2.10.1, it may be possible that by-products of antioxidant action are contributing to a decrease in apparent pH. To confirm this, it would be necessary to first verify that antioxidant content is decreasing (via HPLC) and then to identify antioxidant by-products (via LC-MS/MS).

Following 2 weeks of storage, active and placebo formulations of both Formulations 40 and 42 (containing propyl gallate) were found to be faint yellow at the accelerated temperature, which could be due to antioxidant action (i.e., propyl gallate oxidising in place of the drug) as it the color change was not shown to correlate with any decreases in drug purity. Notably, the formulation with vitamin E (Formulation 41) was clear and colorless. After 4 weeks of storage, Formulation 41 remained colourless (active and placebo), and Formulation 40 and 42 containing propyl gallate were faint yellow at both temperatures (active and placebo). This further suggests that the color change is independent of drug purity and may be related to antioxidant degradation.

At t=0, there were no API crystals in any of the formulations. It should be noted that no particulates of any kind were observed in Formulations 40 or 41 active, however gelling agent particulates were observed in Formulation 42 active and all placebos. It is theorized that this may be due to unhydrated polymer in the formulation. The aliquots of formulation prepared for stability did not appear to exhibit signs of polymer or particulates after 2 weeks of storage at either 25 or 40° C., but were present in the majority of formulations at t=4 week suggesting that those formulations in which polymer was observed at t=0 require longer to achieve full gelling agent hydration.

Each of Formulations 40-42 exhibit good drug chemical stability, and either propyl gallate or vitamin E show to be suitable antioxidants for use in the formulations in place of sodium metabisulfite.

### Example 8: Evaluation of Compositions in the Absence of Antioxidants

Further formulations were created to test the stability of further combinations of excipients, such as compositions that do not contain any antioxidants.

TABLE 20
Model formulations

Composition of formulations (% w/w)
Formulation #

| Excipient | 43 | 44 | 45 | 40 (3%) |
|---|---|---|---|---|
| Compound 1 | 3.028 | 3.028 | 3.028 | 3.000 |
| Deionised water | 45.535 | 45.537 | — | 45.527 |
| pH 5 buffer (41% | — | — | 45.537 | — |
| citrate buffer) | | | | |
| Glycerol | 9.487 | 9.487 | 9.487 | 9.485 |
| Diethylene Glycol | 18.974 | 18.974 | 18.974 | 37.939 |
| Monoethyl Ether | | | | |
| (Transcutol P) | | | | |
| Propylene glycol | 18.974 | 18.974 | 18.974 | — |
| Benzyl alcohol | 2.000 | 2.000 | 2.000 | 2.000 |
| Alpha tocopherol | 0.002 | — | — | — |
| Propyl gallate | — | — | — | 0.050 |
| Hydroxypropyl | 2.00 | 2.00 | 2.00 | 2.00 |
| Methyl Cellulose | | | | |
| (Benecel E4M) | | | | |
| Total | 100.00 | 100.00 | 100.00 | 100.00 |

Formulations 43-45 were based on Formulation 23, but Formulation 43 included alpha tocopherol at 0.002%, Formulation 44 did not include an antioxidant, and Formulation 45 included a pH buffer and no antioxidant. Formulation 40 in Table 20 mirrors that from Example 7 above, but with 3% API.

Analysis of Formulations 40 (3% w/w drug loading), and 43-45 were performed at t=0. Compound 1 recovery was within 100±5% for all formulations, and drug purity from all formulations was >99% area, with the exception of Formulation 45 (with pH 5 buffer instead of water) from which purity of 98.85% was observed. Formulations were clear and colorless apart from Formulation 40 (3%), which was faint yellow and in line with what was observed with the previous Formulation 40 (6%) data after 2 weeks of storage.

### Example 9: Evaluation of Compositions Over Time

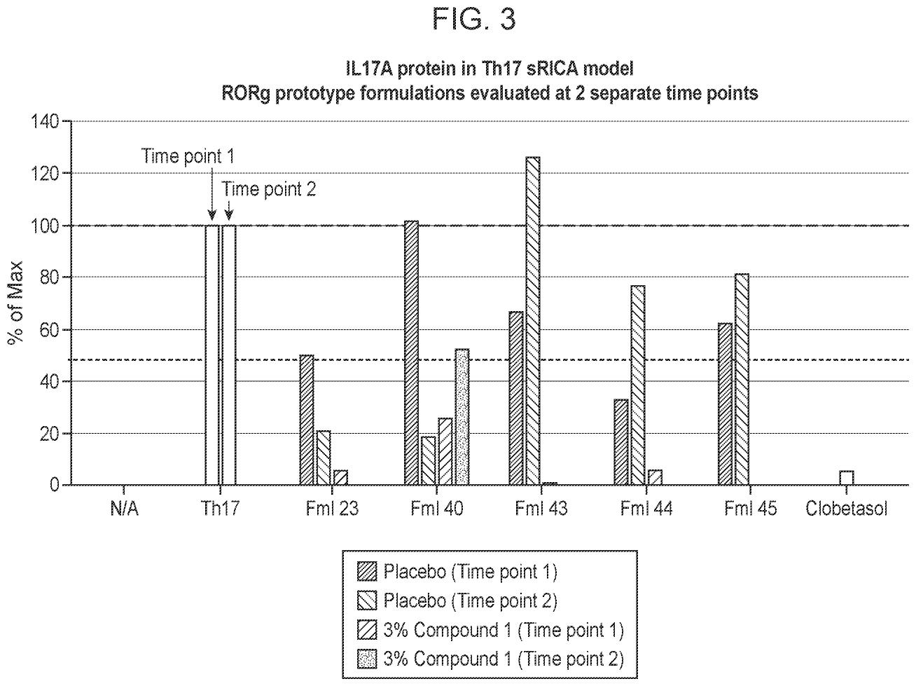

Formulations of Compound 1 were tested in the sRICA model described in Example 2 above at a first time point and a second time point after the formulations were stored for 11 months. The performance of Compound 1 in representative aqueous gel formulations was evaluated. The results for this study are shown in FIG. 3.

As shown, several of the formulations (e.g., Formulations 23, 43, 44 and 45) demonstrated significant ability to inhibit IL17A protein induction in the sRICA model after being stored for 11 months.

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

1. A dermatological composition comprising:
a pharmaceutically effective amount of a RORγt inhibitor;
a dermatologically acceptable carrier;
a humectant; and
a preservative.

2. The dermatological composition according to claim 1, wherein the RORγt inhibitor consists of one or more members selected from the following:
N-(2-((3-fluoro-4-(trimethylsilyl)phenyl)amino)-1-(4-(methoxymethyl)phenyl)-2-oxoethyl)-5-oxopyrrolidine-3-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
N-(1-(4-methoxyphenyl)-2-oxo-2-((4-(trimethylsilyl)phenyl)amino)ethyl)-N-methyl-1,2-oxazole-5-carboxamide;
(2R)—N-(3,5-difluoro-4-(trimethylsilyl)phenyl)-2-(4-(methoxymethyl)phenyl)-2-(((6-oxopyrimidin-1(6H)-yl)acetyl)amino)acetamide;
N-(1-(4-methoxyphenyl)-2-oxo-2-((4-(trimethylsilyl)phenyl)amino)ethyl)-N-methyltetrahydrothiophene-3-carboxamide-1,1-dioxide;
5-((1R)-1-((3,5-difluoro-4-(trimethylsilyl)phenyl)carbamoyl)-6-methoxy-3,4-dihydroisoquinolin-2(1H)-yl)-5-oxopentanoic acid;
5-((1R)-1-((3,5-difluoro-4-(trimethylsilyl)phenyl)carbamoyl)-6-methoxymethyl-3,4-dihydroisoquinolin-2(1H)-yl)-5-oxopentanoic acid;
N-((1R)-2-((3-fluoro-4-(trimethylsilyl)phenyl)amino)-1-(4-methoxyphenyl)-2-oxoethyl)-3-hydroxy-N-methyl-1,2-oxazole-5-carboxamide;
1-acetyl-N-((1R)-2-((3-fluoro-4-(trimethylsilyl)phenyl)amino)-1-(4-(methoxymethyl)phenyl)-2-oxoethyl)piperidine-4-carboxamide;
(2R)—N-(2,5-difluoro-4-(trimethylsilyl)phenyl)-2-(4-(methoxymethyl)phenyl)-2-(((5-methyl-1,3,4-oxadiazol-2-yl)acetyl)amino)acetamide;
(3R)—N-((1R)-2-((3-fluoro-4-(trimethylsilyl)phenyl)amino)-1-(4-(methoxymethyl)phenyl)-2-oxoethyl)-5-oxopyrrolidine-3-carboxamide;
(3R)—N-((1R)-2-((4-tert-butyl-3-fluorophenyl)amino)-1-(4-(methoxymethyl)phenyl)-2-oxoethyl)-5-oxopyrrolidine-3-carboxamide;
(3R)—N-(3-((3,3-difluorocyclobutyl)-methyl)-7-fluoro-1-isopropyl-2,4-dioxo-1,2,3,4-tetrahydro-quinazolin-6-yl)-3-(1-oxo-1,3-dihydro-2H-isoindol-2-yl)-piperidine-1-carboxamide;
(2R)—N-(4-tert-butyl-3-fluorophenyl)-2-(4-(methoxymethyl)phenyl)-2-(((6-oxopyrimidin-1(6H)-yl)acetyl)amino)acetamide;
N-((1R)-2-((4-tert-butyl-3-chlorophenyl)amino)-2-oxo-1-(tetrahydro-2H-pyran-4-yl)ethyl)-3,3,3-trifluoro-2-hydroxypropanamide;
(1R)—N-(3-fluoro-4-(trimethylsilyl)phenyl)-2-((3-hydroxy-1,2-oxazol-5-yl)acetyl)-6-(methoxymethyl)-1,2,3,4-tetrahydroisoquinoline-1-carboxamide;
(2R)-2-(((2,4-dioxo-3,4-dihydropyrimidin-1(2H)-yl)acetyl)amino)-N-(3-fluoro-4-(trimethylsilyl)phenyl)-2-(4-(methoxymethyl)phenyl)acetamide;
(5R)—N-(4-tert-butyl-3-fluorophenyl)-6-((3-hydroxy-1,2-oxazol-5-yl)acetyl)-2-methoxy-5,6,7,8-tetrahydro-1,6-naphthyridine-5-carboxamide;
(5R)—N-(-4-(ethyl(trimethylsilyl)-3,5-difluorophenyl)-6-((3-hydroxy-1,2-oxazol-5-yl)acetyl)-2-methoxymethyl-5,6,7,8-tetrahydro-1,6-naphthyridine-5-carboxamide;
(2R)—N-(4-tert-butyl-3-fluorophenyl)-2-(((3-hydroxy-1,2-oxazol-5-yl)acetyl)amino)-2-(4-(methoxymethyl)phenyl)acetamide;
(1R)-6-ethoxy-N-(7-fluoro-1,1-dimethyl-2,3-dihydro-1H-inden-5-yl)-2-((3-hydroxy-1,2-oxazol-5-yl)acetyl)-1,2,3,4-tetrahydroisoquinoline-1-carboxamide;
N-((1R)-2-((3-fluoro-4-(trimethylsilyl)phenyl)amino)-1-(4-(methoxymethyl)phenyl)-2-oxoethyl)-3-(methylsulfonyl)propenamide;
N-(2-((3-fluoro-4-(trimethylsilyl)phenyl)amino)-1-(4-methoxyphenyl)-2-oxoethyl)-5-oxopyrrolidine-3-carboxamide;
(3R)—N-((1R)-2-((3-fluoro-4-(trimethylsilyl)phenyl)amino)-1-(4-(methoxy)phenyl)-2-oxoethyl)-5-oxopyrrolidine-3-carboxamide;
(3R)—N-((1R)-2-((4-tert-butyl-3,5-difluorophenyl)amino)-1-(4-(methoxymethyl)phenyl)-2-oxoethyl)-5-oxopyrrolidine-3-carboxamide;
(3R)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(3-fluoro-4-(trimethylsilyl)phenyl)-2-((3-hydroxy-1,2-oxazol-5-yl)carbonyl)-6-methoxymethyl-1,2,3,4-tetrahydroisoquinoline-1-carboxamide;
(2R)—N-(4-tert-butyl-3-fluorophenyl)-2-(4-(methoxymethyl)phenyl)-2-(((5-methyl-1,3,4-oxadiazol-2-yl)acetyl)amino)acetamide;
(1R)—N-(4-(1-(cyclopropyl-methoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-(3-chloro-4-cyanophenyl)-N′-(1-ethyl-3-(3-methoxypropyl)-2,4-dioxo-1,2,3,4-tetrahydroquinazolin-6-yl)-3-methylpentanediamide;
(2R)—N-(3,5-difluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(4-tert-butyl-3-fluorophenyl)-2-((3-hydroxy-1,2-oxazol-5-yl)acetyl)-6-methoxymethyl-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-(methoxymethyl)-5,6,7,8-tetrahydro-1,6-naphthyridine-5-carboxamide;
(1R)—N-(7-fluoro-1,1-dimethyl-2,3-dihydro-1H-inden-5-yl)-2-((3-hydroxy-1,2-oxazol-5-yl)carbonyl)-6-(methoxymethyl)-1,2,3,4-tetrahydroisoquinoline-1-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(5R)—N-(3,5-difluoro-4-(trimethylsilyl)phenyl)-4-((3-hydroxy-1,2-oxazol-5-yl)acetyl)-8-methoxy-2,3,4,5-tetrahydro-1,4-benzoxazepine-5-carboxamide;
(1R)—N-(3,5-difluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)carbonyl)-6-methoxy-1,2,3,4-tetrahydro-isoquinoline-1-carboxamide;
N-((1R)-2-((3-fluoro-4-(trimethylsilyl)phenyl)amino)-1-(4-(methoxyphenyl)-2-oxoethyl)-3-hydroxy-1,2-oxazole-5-carboxamide;
(1R)—N-(3-fluoro-4-(1-methoxy-2-methylpropan-2-yl)phenyl)-2-((3-hydroxy-1,2-oxazol-5-yl)carbonyl)-6-methoxy-1,2,3,4-tetrahydroisoquinoline-1-carboxamide;
(1R)—N-(4-(1-ethoxy-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
N-((1R)-2-((3-fluoro-4-(trimethylsilyl)phenyl)amino)-1-(4-(methoxymethyl)phenyl)-2-oxoethyl)-3-hydroxyazetidine-1-carboxamide;
(2R)—N-(3,5-difluoro-4-(trimethylsilyl)phenyl)-2-(((3-hydroxy-1,2-oxazol-5-yl)acetyl)amino)-2-(4-(methoxymethyl)phenyl)acetamide;
(3R)-3-(benzoylamino)-N-(3-(cyclopropylmethyl)-1-isopropyl-2,4-dioxo-1,2,3,4-tetrahydroquinazolin-6-yl)piperidine-1-carboxamide;
(3S)—N-((1R)-2-((4-(2,2-dimethylpropyl)-3-fluorophenyl)amino)-1-(4-(methoxymethyl)phenyl)-2-oxoethyl)-5-oxopyrrolidine-3-carboxamide;
5-((5R)-5-((3-fluoro-4-(trimethylsilyl)phenyl)carbamoyl)-2-methoxy-7,8-dihydro-1,6-naphthyridin-6(5H)-yl)-5-oxopentanoic acid;
5-((1R)-1-((3-fluoro-4-(trimethylsilyl)phenyl)carbamoyl)-6-(methoxymethyl)-3,4-dihydroisoquinolin-2(1H)-yl)-5-oxopentanoic acid;
(1R)—N-(7-fluoro-1,1-dimethyl-2,3-dihydro-1H-inden-5-yl)-2-((3-hydroxy-1,2-oxazol-5-yl)acetyl)-6-(methoxymethyl)-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(1R)—N-(3,5-difluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(R)-2-(2-acetamidoacetamido)-N-(3-fluoro-4-(trimethylsilyl)phenyl)-2-(4-(methoxymethyl)phenyl)acetamide);
(3S)—N-(2-((4-tert-butyl-3-fluorophenyl)amino)-1-(2,3-dihydro-1-benzofuran-5-yl)-2-oxoethyl)-5-oxopyrrolidine-3-carboxamide;
(5R)—N-(4-(ethyl(dimethyl)silyl)-3,5-difluorophenyl)-6-((3-hydroxy-1,2-oxazol-5-yl)carbonyl)-2-(methoxymethyl)-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
(3R)—N-((1R)-2-((3-fluoro-4-(trimethylsilyl)phenyl)amino)-1-(4-methoxyphenyl)-2-oxoethyl)-3-hydroxypyrrolidine-1-carboxamide;
(1R)—N-(3,5-difluoro-4-(1-methoxy-2-methylpropan-2-yl)phenyl)-6-methoxy-2-((3-hydroxy-1,2-oxazol-5-yl)acetyl)-1,2,3,4-tetrahydro-isoquinoline-1-carboxamide;
(3S)—N-((1R)-2-((3-fluoro-4-(trimethylsilyl)phenyl)amino)-1-(1-methyl-1H-indazol-5-yl)-2-oxoethyl)-5-oxopyrrolidine-3-carboxamide;
(3R)—N-(3-(cyclopropylmethyl)-7-fluoro-1-isopropyl-2,4-dioxo-1,2,3,4-tetrahydroquinazolin-6-yl)-3-((2,5-difluorobenzoyl)amino)-piperidine-1-carboxamide;
(1R)—N-(3-cyano-4-(trimethylsilyl)phenyl)-2-((3-hydroxy-1,2-oxazol-5-yl)acetyl)-6-methoxy-1,2,3,4-tetrahydroisoquinoline-1-carboxamide;
N-(7-fluoro-1,1-dimethyl-2,3-dihydro-1H-inden-5-yl)-2-((3-hydroxy-1,2-oxazol-5-yl)carbonyl)-6-methoxy-1,2,3,4-tetrahydroisoquinoline-1-carboxamide;
5-((5R)-5-((4-(ethyl (dimethyl)silyl)-3,5-difluorophenyl)carbamoyl)-2-methoxy-7,8-dihydro-1,6-naphthyridin-6(5H)-yl)-5-oxopentanoic acid;
(2R)-2-(((2,6-dioxo-3,6-dihydropyrimidin-1(2H)-yl)acetyl)amino)-N-(3-fluoro-4-(trimethylsilyl)phenyl)-2-(4-(methoxymethyl)phenyl)acetamide;
(1R)—N-(4-(1-(2,2-difluoroethoxy)-2-methylpropan-2-yl)-3,5-difluorophenyl)-2-((3-hydroxy-1,2-oxazol-5-yl)acetyl)-6-methoxy-1,2,3,4-tetrahydro-isoquinoline-1-carboxamide;
(3S)—N-((1R)-2-((4-tert-butyl-3-fluorophenyl)amino)-1-(4-(methoxymethyl)phenyl)-2-oxoethyl)-5-oxopyrrolidine-3-carboxamide;
(2R)—N-(3,5-difluoro-4-(trimethylsilyl)phenyl)-2-(4-(methoxymethyl)phenyl)-2-(((3-methyl-6-oxopyridazin-1(6H)-yl)acetyl)amino)acetamide;
N-((1R)-2-((3,5-difluoro-4-(trimethylsilyl)phenyl)amino)-1-(4-(methoxymethyl)phenyl)-2-oxoethyl)-3-hydroxy-N-methyl-1,2-oxazole-5-carboxamide;
(3S)—N-(2-((3-fluoro-4-(trimethylsilyl)phenyl)amino)-1-(1-methyl-1H-indazol-5-yl)-2-oxoethyl)-5-oxopyrrolidine-3-carboxamide;
(2R)-2-(((2,5-dioxoimidazolidin-1-yl)acetyl)amino)-N-(3-fluoro-4-(trimethylsilyl)phenyl)-2-(4-(methoxymethyl)phenyl)acetamide;
N-(4-tert-butyl-3-fluorophenyl)-2-((3-hydroxy-1,2-oxazol-5-yl)carbonyl)-6-methoxy-1,2,3,4-tetrahydroisoquinoline-1-carboxamide;
(2R)—N-(3-fluoro-4-(trimethylsilyl)phenyl)-2-(4-(methoxymethyl)phenyl)-2-(((5-methyl-1,3,4-oxadiazol-2-yl)acetyl)amino)acetamide;
(1R)—N-(7-fluoro-1,1-dimethyl-2,3-dihydro-1H-inden-5-yl)-2-((3-hydroxy-1,2-oxazol-5-yl)acetyl)-6-methoxy-1,2,3,4-tetrahydroisoquinoline-1-carboxamide;
N-(2-((3,5-difluoro-4-(trimethylsilyl)phenyl)amino)-2-oxo-1-(tetrahydro-2H-pyran-4-yl)ethyl)-3-hydroxy-N-methyl-1,2-oxazole-5-carboxamide;
(2R)—N-(3-fluoro-4-(trimethylsilyl)phenyl)-2-(4-(methoxymethyl)phenyl)-2-(((methylsulfonyl)acetyl)amino)acetamide;
(1R)—N-(3-fluoro-4-(trimethylsilyl)phenyl)-2-((3-hydroxy-1,2-oxazol-5-yl)acetyl)-6-methoxy-1,2,3,4-tetrahydroisoquinoline-1-carboxamide;
(1R)—N-(4-(1-(2,2-difluoroethoxy)-2-methylpropan-2-yl)-3,5-difluorophenyl)-2-((3-hydroxy-1,2-oxazol-5-yl)carbonyl)-6-methoxy-1,2,3,4-tetrahydroisoquinoline-1-carboxamide;
5-(((1R)-2-((3,5-difluoro-4-(trimethylsilyl)phenyl)amino)-1-(4-(methoxymethyl)phenyl)-2-oxoethyl)amino)-5-oxopentanoic acid;
(2R)—N-(4-tert-butyl-3,5-difluorophenyl)-2-(((3-hydroxy-1,2-oxazol-5-yl)acetyl)amino)-2-(4-(methoxymethyl)phenyl)acetamide;
(5R)—N-(7-fluoro-1,1-dimethyl-2,3-dihydro-1H-inden-5-yl)-6-((3-hydroxy-1,2-oxazol-5-yl)carbonyl)-2-methoxy-5,6,7,8-tetrahydro-1,6-naphthyridine-5-carboxamide;
N-{4-[(3-chloro-4-cyanophenyl)amino]-2-methyl-4-oxobutyl}-9-ethyl-9H-carbazole-3-carboxamide;
(3S)—N-((1R)-2-((3,5-difluoro-4-(trimethylsilyl)phenyl)amino)-1-(4-(methoxymethyl)phenyl)-2-oxoethyl)-5-oxopyrrolidine-3-carboxamide;
(1R)—N-(4-tert-butyl-3-fluorophenyl)-2-((3-hydroxy-1,2-oxazol-5-yl)acetyl)-6-methoxy-1,2,3,4-tetrahydroisoquinoline-1-carboxamide;
(5R)—N-(3-fluoro-4-(trimethylsilyl)phenyl)-6-((3-hydroxy-1,2-oxazol-5-yl)acetyl)-2-methoxy-5,6,7,8-tetrahydro-1,6-naphthyridine-5-carboxamide;
(5R)—N-(7-fluoro-1,1-dimethyl-2,3-dihydro-1H-inden-5-yl)-6-((3-hydroxy-1,2-oxazol-5-yl)acetyl)-2-methoxy-5,6,7,8-tetrahydro-1,6-naphthyridine-5-carboxamide;
N-((1R)-2-((2,5-difluoro-4-(trimethylsilyl)phenyl)amino)-1-(4-methoxyphenyl)-2-oxoethyl)-3-hydroxy-N-methyl-1,2-oxazole-5-carboxamide;
N-(3-fluoro-4-(trimethylsilyl)phenyl)-2-((3-hydroxy-1,2-oxazol-5-yl)carbonyl)-6-methoxy-1,2,3,4-tetrahydroisoquinoline-1-carboxamide;
(1R)—N-(4-tert-butyl-3,5-difluorophenyl)-2-((3-hydroxy-1,2-oxazol-5-yl)acetyl)-6-methoxy-1,2,3,4-tetrahydroisoquinoline-1-carboxamide;
(5R)—N-(3,5-difluoro-4-(trimethylsilyl)phenyl)-6-((3-hydroxy-1,2-oxazol-5-yl)carbonyl)-2-(methoxymethyl)-5,6,7,8-tetrahydro-1,6-naphthyridine-5-carboxamide;
N-((1R)-2-((3-fluoro-4-(trimethylsilyl)phenyl)amino)-1-(4-(methoxymethyl)phenyl)-2-oxoethyl)tetrahydro-2H-pyran-4-carboxamide;
or a stereoisomer, tautomers, or pharmaceutically acceptable salts thereof.

3. The dermatological composition according to claim 1, wherein the carrier comprises water (e.g., deionized water), an alcohol (e.g., ethanol, 2-propanol and n-propanol) and/or a glycol (e.g., polyethylene glycol, e.g., PEG 200, PEG 300, PEG 400).

4. The dermatological composition according to claim 1, wherein the carrier is present in an amount of about 25 wt. % to about 75 wt. %, about 40 wt. % to about 70 wt. %, about 45 wt. % to about 60 wt. %, or about 55 wt. % to about 70 wt. %, based on the total weight of the composition.

5. The dermatological composition according to claim 1, wherein the humectant comprises one or more of glycerol, polypropylene glycol, and/or propylene glycol.

6. The dermatological composition according to claim 1, wherein the humectant comprises or consists of glycerol and propylene glycol.

7. The dermatological composition according to claim 1, wherein the humectant is present in an amount of about 5 wt. % to about 40 wt. %, about 15 wt. % to about 30 wt. %, or about 25 wt. % to about 30 wt. %, based on the total weight of the composition.

8. The dermatological composition according to claim 1, wherein the composition is substantially free from sorbitol.

9. The dermatological composition according to claim 1, wherein the preservative comprises one or more of sodium benzoate, benzyl alcohol, diazolidinyl urea, methylparaben, propylparaben, tetrasodium EDTA, and/or ethylparaben.

10. The dermatological composition according to claim 1, wherein the preservative comprises or consists of sodium benzoate in an amount of about 0.01 wt. % to about 1 wt. %, e.g., about 0.2 wt. %, based on the total weight of the composition.

11. The dermatological composition according to claim 1, further comprising a skin absorption enhancer selected from one or more of a C₁₋₂₀ alkanol (e.g., oleyl alcohol, cetyl alcohol, octyldodecanol, cetostearyl alcohol, benzyl alcohol), a saturated or unsaturated fatty acid ester, a saturated or unsaturated fatty acid ester, a polyoxythylene fatty ether, a polyoxylene fatty acid esters, diethylene glycol monoethyl ether, 1,3-dimethyl-2-imidazolidinone and/or dimethyl isosorbide.

12. The dermatological composition according to claim 1, comprising a skin absorption enhancer in an amount of about 10 wt. % to about 45 wt. %, about 15 wt. % to about 25 wt. %, about 15 wt. % to about 20 wt. %, e.g., about 15 wt. %, about 16 wt. %, about 17 wt. %, about 18 wt. %, about 19 wt. %, or about 20 wt. %, based on the total weight of the composition.

13. The dermatological composition according to claim 1, comprising diethylene glycol monoethyl ether in an amount of about 10 wt. % to about 45 wt. %, about 15 wt. % to about 25 wt. %, about 15 wt. % to about 20 wt. %, e.g., about 15 wt. %, about 16 wt. %, about 17 wt. %, about 18 wt. %, about 19 wt. %, or about 20 wt. %, based on the total weight of the composition.

14. The dermatological composition according to claim 1, wherein the composition comprises propylene glycol and diethylene glycol monoethyl ether in a total amount of greater than about 30 wt. %.

15. The dermatological composition according to claim 1, comprising a viscosity enhancing agent selected from one or more of a cellulose, an acrylate polymer or crosspolymers, or a carbomer.

16. The dermatological composition according to claim 1, comprising a chelating agent selected from one or more of EDTA (e.g., disodium EDTA), disodium edeate, dipotassium edeate, cyclodextrin, trisodium edetate, tetrasodium edetate, citric acid, sodium citrate, gluconic acid and potassium gluconate.

17. The dermatological composition according to claim 1, comprising an antioxidant selected from one or more of butylated hydroxytoluene (BHT), ascorbic acid, propyl gallate, and/or alpha tocopherol (Vitamin E).

18. The dermatological composition according to claim 1, wherein the composition is substantially free from sodium metabisulfite.

19. The dermatological composition according to claim 1, wherein the RORγt inhibitor is present at a concentration of about 0.001 wt. % to about 25 wt. %, based on the total weight of the composition.

20. The dermatological composition according to claim 1, wherein the RORγt inhibitor is present at a concentration of about 0.1 wt. % to about 5 wt. %, based on the total weight of the composition.

21. The dermatological composition according to claim 1, wherein the RORγt inhibitor is present at a concentration of about 2 wt. % to about 3 wt. %, e.g. about 2 wt. % or about 3 wt. %, based on the total weight of the composition.

22. The dermatological composition according to claim 1, wherein the composition has a pH of about 3.5 to about 7.5, about 4 to about 7, about 4.5 to about 6.5 or about 5 to about 6.5.

23. The dermatological composition according to claim 1, wherein the composition is in the form of a cream, a lotion, a foam, an aqueous gel, a non-aqueous gel, a spray or an ointment (e.g., a polyethylene glycol-based ointment).

24. The dermatological composition according to claim 1, wherein the composition is in the form of an aqueous gel.

25. A method for treating an autoimmune disorder, the method comprising topically administering to a subject in need thereof a dermatological composition having a therapeutically effective amount of a RORγt inhibitor according to claim 1.
